(19) United States
(12) Patent Application Publication
(10) Pub. No.: US 2025/0339356 A1
(43) Pub. Date: Nov. 6, 2025

(54) Personal Care Compositions

(71) Applicant: Colgate-Palmolive Company, New York, NY (US)

(72) Inventors: Qiang WU, Hillsborough, NJ (US); Shujiang CHENG, Warren, NJ (US); Nadia SOLIMAN, East Brunswick, NJ (US); Thomas BOYD, Metuchen, NJ (US); Jin NAMKOONG, High Bridge, NJ (US); Sayantani GOSWAMI, Edison, NJ (US)

(73) Assignee: Colgate-Palmolive Company, New York, NY (US)

(21) Appl. No.: 19/270,612

(22) Filed: Jul. 16, 2025

Related U.S. Application Data
(63) Continuation of application No. 17/942,086, filed on Sep. 9, 2022.
(60) Provisional application No. 63/242,704, filed on Sep. 10, 2021.

(51) Int. Cl. A61K 8/44; A61K 8/67; A61K 31/07; A61K 31/185; A61K 31/198; A61K 31/355; A61K 31/375; A61P 17/02; A61P 17/16; A61P 39/06; A61Q 17/04

(52) U.S. Cl. CPC A61K 8/44; A61K 8/447; A61K 8/671; A61K 8/676; A61K 8/678; A61K 31/07; A61K 31/185; A61K 31/198; A61K 31/355; A61K 31/375; A61P 17/02; A61P 17/16; A61P 39/06; A61Q 17/04; A61K 2800/522; A61K 2800/5922

(57) ABSTRACT

Personal care compositions and methods of use are disclosed herein. A personal care composition is provided comprising: a skin protection system comprising: an amino acid complex; and optionally one or more antioxidants; a penetration enhancer; and a cosmetically acceptable carrier.

### CROSS-REFERENCE TO RELATED APPLICATION

This application claims the benefit of priority from U.S. Provisional Application No. 63/242,704, entitled “PERSONAL CARE COMPOSITIONS” and filed Sep. 10, 2021, the contents of which are hereby incorporated herein in their entirety.

### BACKGROUND

As the outermost layer, skin is a key barrier protecting the internal organs from external aggressors. Ultraviolet (UV) light is one external aggressor that could cause problems including the death of skin cells and/or damage to their critical components, such as the nucleus. Accumulation of UV damage of the DNA of a cell can lead to skin cancer.

UV light also contributes to aging by causing free radicals to form in the skin. Free radicals include, e.g., singlet oxygen, hydroxyl radical, the superoxide anion, nitric oxide and hydrogen radicals. Free radicals attack DNA, membrane lipids and proteins, generating carbon radicals. These in turn react with oxygen to produce a peroxyl radical that can attack adjacent fatty acids to generate new carbon radicals. This cascade leads to a chain reaction producing lipid peroxidation products. Damage to the cell membrane results in loss of cell permeability, increased intercellular ionic concentration, and decreased ability to excrete or detoxify waste products. The end result is a loss of skin elasticity and the appearance of wrinkles. This process is commonly referred to as photo-aging.

Accordingly, there is an ongoing need for personal care products that can protect the skin from UV light and facilitate the recovery of skin damaged by such UV light.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to personal care compositions for improving the health and/or appearance of skin. In accordance with one aspect, provided is a personal care composition including: a skin protection system comprising an amino acid complex and, optionally, one or more antioxidants; a penetration enhancer; and a cosmetically acceptable carrier.

The amino acid complex may comprise an amino acid selected from: taurine; arginine; glycine; serine; lysine; and a combination of two or more thereof. In some embodiments, the amino acid complex comprises: taurine and arginine; wherein the weight ratio of taurine: arginine is from about 1: 10 to about 10: 1. For example, the amino acid complex may comprise: taurine and arginine; wherein the weight ratio of taurine: arginine is from about 1: 5 to about 5:1, optionally from about 1: 2 to about 2: 1, or about 2: 1, or 65: 34. Additionally or alternatively, the amino acid complex of the personal care composition may comprise: taurine; arginine; and glycine, wherein the weight ratio of taurine: arginine: glycine is from about 1: 1: 1 to about 100: 50: 1. In certain embodiments, the amino acid complex comprises: taurine; arginine; and glycine, wherein the weight ratio of taurine: arginine: glycine is about 65: 34: 1.

The antioxidant of the personal care composition may be selected from: sulfhydryl compounds; lipoic acid; dihydrolipoic acid; resveratrol; lactoferrin; ascorbic acid; butylated hydroxytoluene; retinoids; tocopherols; tocotrienols; ubiquinone; vitamin E; vitamin C; vitamin A; a derivative thereof; and a combination of two or more thereof. For instance, the antioxidant may be selected from: vitamin E; vitamin C; vitamin A; a derivative thereof; and a combination of two or more thereof.

The personal care compositions generally include a penetration enhancer. Examples of penetration enhancers include ethanol, dimethyl sulfoxide, dimethyl isosorbide, isopropyl myristate; propylene glycol; and a combination of two or more thereof.

The personal care composition may comprise a hydrophobic component. The hydrophobic component may include an oil, a wax, a silicone, or a mixture thereof. The oil may be a plant-based oil. For example, the oil may be a plant-based oil selected from sunflower oil, corn oil, soybean oil, marrow oil, grapeseed oil, sesame seed oil, hazelnut oil, apricot oil, macadamia oil, araba oil, coriander oil, castor oil, avocado oil, jojoba oil, shea butter oil, or a combination of two or more thereof.

The hydrophobic component may include a wax comprising a linear or branched hydrocarbon, optionally of mineral or synthetic origin. For example, the linear or branched hydrocarbon may be selected from volatile or non-volatile liquid paraffins and derivatives thereof, petroleum jelly, polydecenes, isohexadecane, isododecane, hydrogenated polyisobutene, a mixture of n-undecane (C₁₁) and of n-tridecane (C₁₃), or a combination of two or more thereof.

The silicone of the hydrophobic component, when present, may comprise a volatile silicone oil, optionally selected from cyclopolydimethylsiloxanes (cyclomethicones), such as cyclohexadimethylsiloxane and cyclopentadimethylsiloxane. The silicone may include a polydimethylsiloxane, optionally having from 2 to 24 carbon atoms. In some embodiments, the silicone comprises a phenyl silicone, optionally selected from phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxy silicates, polymethylphenylsiloxanes, and a combination of two or more thereof.

The personal care composition may include an acceptable carrier that comprises a hydrophilic component. For example, the hydrophilic component may include a monoalcohol, a fatty alcohol, a fatty ether, a fatty ester, a polyol, a glycol, or a combination of two or more thereof. In some cases, the hydrophilic component comprises ethyl alcohol, isopropyl alcohol, propyl alcohol, benzyl alcohol, phenylethyl alcohol, ethylene glycol, propylene glycol, butylene glycol, hexylene glycol, propane diol, glycerin, ethers of glycol, or a combination of two or more thereof.

In some embodiments, the personal care composition comprises a cosmetically acceptable active agent selected from an anti-acne agent, a shine control agent, an anti-microbial agent, an anti-inflammatory agent, an anti-mycotic agent, an anti-parasite agent, an external analgesic, a keratolytic agent, a surfactant, a moisturizer, a nutrient, a vitamin, an energy enhancer, an anti-perspiration agents, an astringent, a deodorant, a firming agent, an anti-callous agent, an agent for skin conditioning, and a combination of two or more thereof.

In accordance with another aspect, a method is provided for protecting a skin surface from ultraviolet light; treating, inhibiting or preventing sunburn; and/or ameliorating a symptom associated with excessive sun exposure, the method comprising: applying a personal care composition to a skin surface of a subject in need thereof. The method may be used for symptom(s) associated with excessive sun exposure selected from: inflammation; itch; hypersensitivity; peeling; rash; chills; burning; pain; blisters; sores; flaking; and a combination of two or more thereof.

In accordance with a further aspect, the use of a composition comprising an amino acid complex is provided for protecting a skin surface from ultraviolet light; treating, inhibiting or preventing sunburn; and/or ameliorating a symptom associated with excessive sun exposure. The use of a composition may be for symptom(s) associated with excessive sun exposure selected from: inflammation; itch; hypersensitivity; peeling; rash; chills; burning; pain; blisters; sores; flaking; and a combination of two or more thereof.

The use of the composition may include a composition having an amino acid selected from: taurine; arginine; glycine; serine; lysine; and a combination of two or more thereof. In some cases, the use of a composition includes an amino acid complex comprising an amino acid selected from: taurine; arginine; glycine; and a combination of two or more thereof. In some embodiments, the use of the composition includes a composition wherein the amino acid complex comprises: taurine and arginine, wherein the weight ratio of taurine: arginine is from about 1:10 to about 10:1. For example, the amino acid complex comprises: taurine and arginine, wherein the weight ratio of taurine: arginine is from about 1:5 to about 5:1, optionally from about 1:2 to about 2:1, or about 2:1, or about 65:34.

The use of the composition may include an amino acid complex that comprises: taurine; arginine; and glycine, wherein the weight ratio of taurine: arginine: glycine is from about 1:1: 1 to about 100:50: 1. For example, the amino acid complex may comprise: taurine; arginine; and glycine, wherein the weight ratio of taurine: arginine: glycine is about 65:34: 1.

Additionally or alternatively, the use of the composition may further comprise a cosmetically acceptable carrier, such as a surfactant system comprising a non-ionic surfactant. The non-ionic surfactant may be selected from: an alkyl polyglucoside; a polysorbate; and a combination thereof. In some embodiments, the use of the composition may further comprise a moisturizing agent; a sunscreen active; an antioxidant; a penetration enhancer; and a combination of two or more thereof. The moisturizing agent may comprise a silicone, a vitamin, an extract, and a combination thereof.

### BRIEF DESCRIPTION OF THE DRAWINGS

The features, and advantages of the invention will be apparent from the following more detailed description of certain embodiments of the invention and as illustrated in the accompanying drawings in which:

FIG. 1 is a graph of the cell viability at various concentrations of amino acid mixtures after 24 hours in accordance with aspects of the invention;

FIG. 2 is a graph of the cell viability at various concentrations of amino acid mixtures after 48 hours in accordance with aspects of the invention;

FIGS. 3A and 3B are graphs of the fibroblasts' cell contractile force in accordance with aspects of the invention;

FIG. 4 is a graph of collagen I synthesis in the fibroblasts as measured by ELISA in accordance with aspects of the invention;

FIGS. 5A-l C are graphs of histamine, an allergen biomarker, expression over time in ex vivo explants in accordance with aspects of the invention;

FIGS. 6A-6C are graphs of TNFα expression over time in ex vivo explants in accordance with aspects of the invention;

FIGS. 7A-7C are graphs of MMP-1 expression over time in ex vivo explants in accordance with aspects of the invention;

FIG. 8 is a bar graph of IL-1α expression, a skin irritation biomarker, in artificial human skin in accordance with aspects of the invention;

FIG. 9 is an image of thymine dimer presence in artificial skin in accordance with aspects of the invention;

FIG. 10A is an image of keratinocyte proliferation biomarker in artificial skin in accordance with aspects of the invention;

FIG. 10B is a bar graph of the keratinocyte proliferation biomarker in the artificial skin of FIG. 10A;

FIG. 11 is a bar graph of MKI67 gene expression in the artificial skin in accordance with aspects of the invention;

FIG. 12 is a bar graph of filaggrin expression, a skin barrier marker, in the artificial skin as assessed using QPCR in accordance with aspects of the invention;

FIG. 13 is a bar graph of filaggrin expression, a skin barrier marker, in the artificial skin as assessed using ELISA in accordance with of the invention;

FIG. 14 is a bar graph of NFE2L2 expression, antioxidant regulator gene, in the artificial skin as assessed in accordance with aspects of the invention;

FIGS. 15A-15E are images of Ki67 expression for artificial skin receiving an exemplary personal care composition or a comparative composition in accordance with aspects of the invention;

FIG. 16 is a bar graph of Ki67 expression for artificial skin receiving an exemplary personal care composition or a comparative composition in accordance with aspects of the invention; and

FIG. 17 is a bar graph of IL-1α expression for artificial skin receiving an exemplary personal care composition or a comparative composition in accordance with aspects of the invention.

It should be understood that the various aspects are not limited to the compositions, arrangements, and instrumentality shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “°” refers to a degree, such as a temperature degree or a degree of an angle. The symbols “h”, “min”, “mL”, “nm”, “μm” means hour, minute, milliliter, nanometer, and micrometer, respectively. The abbreviation “UV-VIS” as referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “−”, “=” and “≡” mean single bond, double bond, and triple bond, respectively.

“Volatile”, as used herein, means having a flash point of less than about 100° C. “Non-volatile”, as used herein, means having a flash point of greater than about 100° C.

“Cosmetically acceptable” means that the item in question is compatible with a keratinous substrate, such as skin. For example, a “cosmetically acceptable carrier” means a carrier that is compatible with a keratinous substrate, such as skin.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the instant disclosure can be free or essentially free of all components and elements positively recited throughout the instant disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds may be characterized as both an emulsifier and a surfactant. If a particular personal care composition includes both an emulsifier and a surfactant, a compound that may be characterized as both an emulsifier and a surfactant will serve only as either an emulsifier or a surfactant-not both.

For readability purposes, the chemical functional groups are in their adjective form; for each of the adjectives, the word “group” is assumed. For example, the adjective “alkyl” without a noun thereafter, should be read as “an alkyl group”.

Aspects of the invention relate to personal care compositions and methods of use thereof. A personal care composition is typically provided comprising: a skin protection system having an amino acid complex, and optionally one or more antioxidants, a penetration enhancer, and a cosmetically acceptable carrier.

The personal care compositions include an amino acid complex in an amount that may vary, but typically is in a range from about 0.1 to about 10 wt. %, based on the total weight of the personal care composition. For instance, the amount of amino acid complex in the personal care composition may be from about 0.1 to about 10 wt. %, about 0.1 to about 8 wt. %, about 0.1 to about 6 wt. %, about 0.1 to about 4 wt. %, about 0.1 to about 3 wt. %, about 0.1 to about 2 wt. %, about 0.1 to about 1 wt. %; about 0.5 to about 10 wt. %, about 0.5 to about 8 wt. %, about 0.5 to about 6 wt. %, about 0.5 to about 4 wt. %, about 0.5 to about 3 wt. %, about 0.5 to about 2 wt. %, about 0.5 to about 1 wt. %; about 1 to about 10 wt. %, about 1 to about 8 wt. %, about 1 to about 6 wt. %, about 1 to about 4 wt. %, about 1 to about 3 wt. %, about 1 to about 2 wt. %, including ranges and subranges thereof, based on the total weight of the personal care composition.

The amino acid complex may comprise an amino acid selected from: taurine; arginine; glycine; serine; lysine; and a combination of two or more thereof. Preferably, the amino acid complex includes taurine; arginine; glycine; or a combination of two or more thereof. The amino acid complex may comprise two or more, three or more, four or more, five or more, six or more, seven or more, or eight or more amino acids. In at least one embodiment, the amino acid complex is formed of two, three, four, five, six, or any range or subrange therebetween, of amino acids.

The amino acid complex may have taurine and arginine in amounts, wherein the weight ratio of taurine to arginine is from about 1:10 to about 10:1. For instance, the weight ratio of taurine to arginine may be from about 1:5 to about 5:1, optionally from about 1:2 to about 2:1, or about 2:1, or 65:34. Additionally or alternatively, the amino acid complex includes taurine, arginine, and glycine in amounts, wherein the weight ratio of taurine: arginine: glycine is from about 1:1: 1 to about 100:50: 1, optionally from about 1:1: 1 to about 76:42: 1. In one embodiment, the weight ratio of taurine: arginine: glycine is about 65:34: 1.

The personal care compositions may, in some instances, comprise one or more antioxidants. The amount of antioxidant present in the personal care composition may be from about 0.01 to about 10 wt. %, based on the total weight of the personal care composition. In some embodiments, the amount of antioxidants in the personal care composition may be from about 0.1 to about 10 wt. %, about 0.1 to about 8 wt. %, about 0.1 to about 6 wt. %, about 0.1 to about 4 wt. %, about 0.1 to about 3 wt. %, about 0.1 to about 2 wt. %, about 0.1 to about 1 wt. %; about 0.5 to about 10 wt. %, about 0.5 to about 8 wt. %, about 0.5 to about 6 wt. %, about 0.5 to about 4 wt. %, about 0.5 to about 3 wt. %, about 0.5 to about 2 wt. %, about 0.5 to about 1 wt. %; about 1 to about 10 wt. %, about 1 to about 8 wt. %, about 1 to about 6 wt. %, about 1 to about 4 wt. %, about 1 to about 3 wt. %, about 1 to about 2 wt. %, including ranges and subranges thereof, based on the total weight of the personal care composition.

Examples of antioxidants include, but are not limited to, water-soluble antioxidants such as sulfhydryl compounds and their derivatives (e.g., sodium metabisulfite and N-acetylcysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants that may be suitable for use in the personal care compositions include, but are not limited to, butylated hydroxytoluene, retinoids (e.g., retinol and retinyl palmitate), tocopherols (e.g., tocopherol acetate), tocotrienols, and ubiquinone. In at least one embodiment, the personal care composition includes an antioxidant selected from: sulfhydryl compounds; lipoic acid; dihydrolipoic acid; resveratrol; lactoferrin; ascorbic acid; butylated hydroxytoluene; retinoids; tocopherols; tocotrienols; ubiquinone; vitamin E; vitamin C; vitamin A; a derivative thereof; and a combination of two or more thereof. Preferably, the antioxidant(s) comprises or is selected from: vitamin E; vitamin C; vitamin A; a derivative thereof; and a combination of two or more thereof.

The personal care compositions comprise one or more penetration enhancer(s) in an amount that may vary, but typically in a range from about 0.01 to about 10 wt. %, based on the total weight of the personal care composition. For instance, the amount of penetration enhancer present in the personal care composition may be from about 0.01 to about 10 wt. %, about 0.01 to about 8 wt. %, about 0.01 to about 6 wt. %, about 0.01 to about 4 wt. %, about 0.01 to about 3 wt. %, about 0.01 to about 2 wt. %, about 0.01 to about 1 wt. %; about 0.05 to about 10 wt. %, about 0.05 to about 8 wt. %, about 0.05 to about 6 wt. %, about 0.05 to about 4 wt. %, about 0.05 to about 3 wt. %, about 0.05 to about 2 wt. %, about 0.05 to about 1 wt. %; about 0.1 to about 10 wt. %, about 0.1 to about 8 wt. %, about 0.1 to about 6 wt. %, about 0.1 to about 4 wt. %, about 0.1 to about 3 wt. %, about 0.1 to about 2 wt. %, about 0.1 to about 1 wt. %; about 0.5 to about 10 wt. %, about 0.5 to about 8 wt. %, about 0.5 to about 6 wt. %, about 0.5 to about 4 wt. %, about 0.5 to about 3 wt. %, about 0.5 to about 2 wt. %, about 0.5 to about 1 wt. %; about 1 to about 10 wt. %, about 1 to about 8 wt. %, about 1 to about 6 wt. %, about 1 to about 4 wt. %, about 1 to about 3 wt. %, about 1 to about 2 wt. %, including ranges and subranges thereof, based on the total weight of the personal care composition.

Suitable penetration enhancers include ethanol, dimethyl sulfoxide, dimethyl isosorbide, isopropyl myristate; propylene glycol; and a combination of two or more thereof. The personal care composition may include one or more penetration enhancers, such as 2, 3, 4, 5, 6, or 7 penetration enhancers or ranges and subranges thereof. For instance, the personal care composition may include one or more penetration enhancer(s) selected from ethanol, isopropyl myristate, propylene glycol, and a combination of two or more thereof.

The personal care compositions typically include a cosmetically acceptable carrier. The amount of cosmetically acceptable carrier present in the personal care composition may be from about 5 wt. % to about 98 wt. %, based on the total weight of the personal care composition. In some instances, the amount of cosmetically acceptable carrier present in the personal care composition is from about 5 to about 98 wt. %, about 25 to about 98 wt. %, about 45 to about 98 wt. %, about 65 to about 98 wt. %, about 75 to about 98 wt. %, about 85 to about 98 wt. %, about 90 to about 98 wt. %, about 95 to about 98 wt. %; about 5 to about 85 wt. %, about 25 to about 85 wt. %, about 45 to about 85 wt. %, about 65 to about 85 wt. %, about 75 to about 85 wt. %; about 5 to about 75 wt. %, about 25 to about 75 wt. %, about 45 to about 75 wt. %, about 65 to about 75 wt. %; about 5 to about 65 wt. %, about 25 to about 65 wt. %, about 45 to about 65 wt. %; about 5 to about 55 wt. %, about 25 to about 55 wt. %, about 45 to about 55 wt. %; or about 5 to about 45 wt. %, or about 25 to about 45 wt. %, including ranges and subranges thereof, based on the total weight of the personal care composition.

The phrase “cosmetically acceptable” refers to a material that is compatible with skin and/or hair. The cosmetically acceptable carrier may include, e.g., water and/or water soluble solvents. Non-limiting examples of cosmetically acceptable carriers include glycerin, C₁₋₄ alcohols, organic solvents, fatty alcohols, fatty ethers, fatty esters, polyols, glycols, vegetable oils, mineral oils, liposomes, laminar lipid materials, water, or any combinations of two or more thereof.

Examples of organic solvents include, but are not limited to, monoalcohols and polyols. Exemplary monoalcohols include ethyl alcohol, isopropyl alcohol, propyl alcohol, benzyl alcohol, phenylethyl alcohol, and combinations thereof. The organic solvents may comprise glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monocthyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

The cosmetically acceptable carrier may comprise or consist of a hydrophobic component, a hydrophilic component, or a combination thereof. In at least one embodiment, the cosmetically acceptable carrier consists solely of hydrophilic ingredients or hydrophobic ingredients.

The hydrophilic component may include a monoalcohol, a fatty alcohol, a fatty ether, a fatty ester, a polyol, a glycol, or a combination of two or more thereof. For instance, the hydrophilic component may comprise ethyl alcohol, isopropyl alcohol, propyl alcohol, benzyl alcohol, phenylethyl alcohol, ethylene glycol, propylene glycol, butylene glycol, hexylene glycol, propane diol, glycerin, ethers of glycol, or a combination of two or more thereof.

Additionally or alternatively, the cosmetically acceptable carrier may include a hydrophobic component that comprises an oil, a wax, a silicone, or a mixture thereof. The oil or wax may be a linear or branched hydrocarbon, optionally of mineral or synthetic origin. The linear or branched hydrocarbon may be selected from volatile or non-volatile liquid paraffins and derivatives thereof, petroleum jelly, polydecenes, isohexadecane, isododecane, hydrogenated polyisobutene, a mixture of n-undecane (C₁₁) and of n-tridecane (C₁₃), and a combination of two or more thereof.

In some cases, the oil is a plant-based oil. For example, the oil may be a plant-based oil selected from sunflower oil, corn oil, soybean oil, marrow oil, grapeseed oil, sesame seed oil, hazelnut oil, apricot oil, macadamia oil, araba oil, coriander oil, castor oil, avocado oil, jojoba oil, shea butter oil, or a combination of two or more thereof.

The hydrophobic component may include one or more volatile or non-volatile silicones. For instance, a volatile silicone oil may be included in the personal care composition, optionally selected from cyclopolydimethylsiloxanes (cyclomethicones), such as cyclohexadimethylsiloxane and cyclopentadimethylsiloxane.

The silicone may have from 2 to 24 carbon atoms. For example, the silicone may be a polydimethylsiloxane, optionally having from 2 to 24 carbon atoms, from 2 to 20 carbon atoms, or from 6 to 20 carbon atoms. In some instances, the silicone comprises a phenyl silicones, optionally selected from phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxy silicates, polymethylphenylsiloxanes, and a combination of two or more thereof.

In accordance with another aspect of the invention, the personal care composition may be formulated to be a sunscreen composition. For example, the personal care composition comprises a skin protection system having one or more sunscreen agent(s), an amino acid complex, a penetration enhancer, optionally one or more antioxidants, and optionally a cosmetically acceptable carrier.

The one or more sunscreen agent(s) may be organic UV filtering agent(s) and/or mineral UV filtering agent(s). Mineral UV filtering agents are compounds that do not include any carbon atoms in their chemical structures and are capable of screening out or absorbing UV radiation between 280 and 400 nm. Non-limiting examples of mineral UV filtering agent include treated or untreated metal oxides such as, for example, pigments or nanopigments of titanium oxide (amorphous or crystallized in rutile and/or anatase form), of iron oxide, of zinc oxide, of zirconium oxide, or of cerium oxide. In some embodiments, the mineral UV filtering agents are selected from titanium dioxide zinc oxide, cerium oxide, or a combination of two or more thereof.

The mineral UV filtering agents may selected from treated mineral oxides. Treated mineral oxides are metal oxides that have undergone one or more surface treatments of chemical, electronic, mechanochemical and/or mechanical nature with compounds, such as amino acids, beeswax, fatty acids, fatty alcohols, anionic surfactants, lecithins, sodium, potassium, zinc, iron or aluminium salts of fatty acids, metal (titanium or aluminium) alkoxides, polyethylene, silicones, proteins (collagen or elastin), alkanolamines, silicon oxides, metal oxides, sodium hexametaphosphate, alumina or glycerol.

Mixtures of metal oxides may also be used, especially of titanium dioxide and of cerium dioxide. Examples of mixtures of metal oxides include silica-coated equal-weight mixture of titanium dioxide and of cerium dioxide; alumina, silica and silicone-coated mixtures of titanium dioxide and zinc dioxide; or alumina, silica, and glycerol-coated mixture of titanium dioxide and zinc dioxide.

The mineral UV filtering agents may have a mean particle size from about 5 nm to about 25 um, about 10 nm to about 10 um, or about 15 nm to about 5 μm. The mineral UV filtering agents may be nano-pigments having a mean particle size of about 5 nm to about 100 nm, about 5 nm to about 75 nm, or about 10 nm to 50 nm. Larger particles sizes for the mineral UV filtering agents may also be useful, for example, about 1 μm to about 25 μm, about 5 μm to about 20 μm, about 10 μm to about 15 μm, or any range or subrange thereof.

The total amount of mineral UV filtering agents in the personal care composition can vary, but is typically about 1 to about 30 wt. %, based on the total weight of the personal care composition. For example, the mineral UV filter agent(s) may be present in the personal care composition in an amount from about 1 to about 30 wt. %, about 1 to about 25 wt. %, about 1 to about 20 wt. %, about 1 to about 15 wt. %, about 1 to about 10 wt. %, about 1 to about 7 wt. %, about 1 to about 5 wt. %; from about 5 to about 30 wt. %, about 5 to about 25 wt. %, about 5 to about 20 wt. %, about 5 to about 15 wt. %, about 5 to about 10 wt. %; from about 10 to about 30 wt. %, about 10 to about 25 wt. %, about 10 to about 20 wt. %, about 10 to about 15 wt. %; from about 15 to about 30 wt. %, about 15 to about 25 wt. %, about 15 to about 20 wt. %; from about 20 to about 30 wt. %, about 20 to about 25 wt. %; from about 25 to about 30 wt. %, including ranges and subranges thereof, based on the total weight of the personal care composition. In some instances, the total amount of mineral UV filtering agents may be about 1 to about 25 wt. %, about 1 to about 20 wt. %, about 1 to about 15 wt. %, about 1 to about 10 wt. %, about 5 to about 30 wt. %, about 5 to about 25 wt. %, about 5 to about 20 wt. %, about 5 to about 5 wt. %, about 5 to about 10 wt. %, based on the total weight of the personal care composition.

The personal care composition may, additionally or alternatively, include organic UV filtering agent(s). The organic UV filters may be active in the UVA and/or UVB region. The organic UV filter may be hydrophilic and/or lipophilic. The organic UV filter may be solid or liquid. The terms “solid” and “liquid” mean solid and liquid, respectively, at 25° C. under 1 atm. The organic UV filter can be selected from the group consisting of anthranilic compounds; dibenzoylmethane compounds; cinnamic compounds; salicylic compounds; camphor compounds; benzophenone compounds; diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures of two or more thereof.

Mention may be made, as examples of the organic UV filter(s), of: anthranilic compounds, such as menthyl anthranilate; dibenzoylmethane compounds, such as butyl methoxydibenzoylmethane and isopropyl dibenzoylmethane; cinnamic compounds, such as ethylhexyl methoxycinnamate, isopropyl methoxycinnamate, isopropoxy methoxycinnamate, isoamyl methoxycinnamate, cinoxate (2-cthoxyethyl-4-methoxy cinnamate), DEA methoxycinnamate, diisopropyl methylcinnamate, and glyceryl ethylhexanoate dimethoxycinnamate; salicylic compounds, such as homosalate (homomentyl salicylate), ethylhexyl salicylate, glycol salicylate, butyloctyl salicylate, phenyl salicylate, dipropyleneglycol salicylate, and TEA salicylate; camphor compounds, such as benzylidenecamphor derivatives (e.g., 3-benzylidene camphor), 4-methylbenzylidene camphor, benzylidene camphor sulfonic acid, camphor benzalkonium methosulfate, terephthalylidene dicamphor sulfonic acid, and polyacrylamidomethyl benzylidene camphor; benzophenone compounds, such as benzophenone-1 (2,4-dihydroxybenzophenone), benzophenone-2 (Tetrahydroxybenzophenone), Benzophenone-3 (2-hydroxy-4-methoxybenzophenone) or oxybenzone, benzophenone-4 (hydroxymethoxy benzophonene sulfonic acid), benzophenone-5 (Sodium hydroxymethoxy benzophenone Sulfonate), benzophenone-6 (dihydroxy dimethoxy benzophenone), benzophenone-8, benzophenone-9 (Disodium dihydroxy dimethoxy benzophenonedisulfonate), and benzophenone-12, and n-hexyl 2-(4-diethylamino-2-hydroxybenzoyl) benzoate (UVINUL A+ by BASF); diphenylacrylate compounds, such as octocrylene, and ctocrylene; triazine compounds, such as diethylhexyl butamido triazone, 2,4,6-tris(dincopentyl 4″-aminobenzalmalonate)-s-triazine, bis-ethylhexyloxyphenol methoxyphenyl triazine, and ethylhexyl triazone; benzotriazole compounds, such as phenylbenzotriazole derivatives (e.g., 2-(2H-benzotriazole-2-yl)-6-dodecyl-4-methylpheno, branched and linear); benzalmalonate compounds, such as dineopentyl 4′-methoxybenzalmalonate, and polyorganosiloxane comprising benzalmalonate functional groups (e.g., polysilicone-15); benzimidazole compounds, such as phenylbenzimidazole derivatives (e.g., phenylbenzimidazole sulfonic acid), and disodium phenyl dibenzimidazole tetrasulfonate; imidazoline compounds, such as ethylhexyl dimethoxybenzylidene dioxoimidazoline propionate; bis-benzoazolyl compounds; para-aminobenzoic acid compounds, such as PABA (p-aminobenzoic acid), ethyl PABA, Ethyl dihydroxypropyl PABA, pentyl dimethyl PABA, ethylhexyl dimethyl PABA, glyceryl PABA, and PEG-25 PABA; methylene bis-(hydroxyphenylbenzotriazol) compounds, such as 2,2′-methylenebis[6-(2H-benzotriazol-2-yl)-4-methyl-phenol], 2,2′-methylenebis[6-(2H-benzotriazol-2-yl)-4-(1,1,3,3-tetramethylbutyl) phenol], and drometrizole trisiloxane; and benzoxazole compounds, such as 2,4-bis[5-1(dimethylpropyl) benzoxazol-2-yl-(4-phenyl) imino]-6-(2-ethylhex-yl) imino-1,3,5-triazine.

In some embodiments the organic UV filter agent(s) may be selected from the group consisting of: butyl methoxydibenzoylmethane, ethylhexyl methoxycinnamate, homosalate, ethylhexyl salicylate, phenylbenzimidazole sulfonic acid, benzophenone-3, benzophenone-4, benzophenone-5, n-hexyl 2-(4-dicthylamino-2-hydroxybenzoyl) benzoate, I, r-(1,4-piperazinediyl) bis[]-[2-[4-(dicthylamino)-2-hydroxybenzoyl] phenyl]--methanone 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s-triazinc, 2,4-bis-(n-butyl 4′-aminobenzalmalonate)-6-[(3-{1,3,3,3-tetramethyl-1-[(trimethylsilyloxy]-disiloxanyl}propyl)amino]-s-triazinc, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dincopentyl 4′-methoxybenzalmalonate, I,I-dicarboxy (2,2′-dimethylpropyl)-4,4-diphenylbutadiene, 2,4-bis[5-1 (dimethylpropyl)benzoxazol-2-yl-(4-phenyl)imino]-6-(2-ethylhexyl)imino-1,-3,5-triazine, camphor benzylkonium methosulfate, and mixtures thereof.

The total amount of organic UV filter agents in the personal care compositions can vary, but is typically about 1 to about 30 wt. %, based on the total weight of the personal care composition. For example, the organic UV filter agent(s) may be present in the personal care composition in an amount from about 1 to about 30 wt. %, about 1 to about 25 wt. %, about 1 to about 20 wt. %, about 1 to about 15 wt. %, about 1 to about 10 wt. %, about 1 to about 7 wt. %, about 1 to about 5 wt. %; from about 5 to about 30 wt. %, about 5 to about 25 wt. %, about 5 to about 20 wt. %, about 5 to about 15 wt. %, about 5 to about 10 wt. %; from about 10 to about 30 wt. %, about 10 to about 25 wt. %, about 10 to about 20 wt. %, about 10 to about 15 wt. %; from about 15 to about 30 wt. %, about 15 to about 25 wt. %, about 15 to about 20 wt. %; from about 20 to about 30 wt. %, about 20 to about 25 wt. %; from about 25 to about 30 wt. %, including ranges and subranges thereof, based on the total weight of the personal care composition. In some instances, the total amount of organic UV filter agents may be about 1 to about 25 wt. %, about 1 to about 20 wt. %, about 1 to about 15 wt. %, about 1 to about 10 wt. %, about 5 to about 30 wt. %, about 5 to about 25 wt. %, about 5 to about 20 wt. %, about 5 to about 5 wt. %, about 5 to about 10 wt. %, based on the total weight of the personal care composition.

The personal care composition may include one or more film-former(s). In certain embodiments, the combination of film-former(s) with sunscreen agents, an amino acid complex, optionally one or more antioxidants, optionally a penetration enhancer, and optionally a cosmetically acceptable carrier promotes even coverage of the sunscreen agents. Preferably, the film-former(s) are selected and included in amounts such that the personal care composition is water resistant. The film-former is typically a hydrophobic material that imparts film forming and/or waterproofing characteristics. Examples of film-formers include polyethylene, synthetic wax, acrylates/acrylamide copolymer, acrylates copolymer, acrylates/C₁₂-C₂₂ alkylmethacrylate copolymer, polyethylene, waxes, VP/dimethiconylacrylate/polycarbamylpolyglycol ester, butylated PVP, PVP/hexadecene copolymer, octadecene/MA copolymer, PVP/eicosene copolymer, tricontanyl PVP, Brassica Campestris/Aleuritis Fordi Oil copolymer, decamethyl cyclopentasiloxane (and) trimethylsiloxysilicate, and mixtures thereof. In some cases, the film former is acrylates/C12-C22 alkylmethacrylate copolymer.

The amount of film-former(s) in the personal care composition may vary, but typically ranges from about 1 to about 30 wt. %, based on the total weight of the personal care composition. For example, the film-former(s) may be present in the personal care composition in an amount from about 1 to about 30 wt. %, about 1 to about 25 wt. %, about 1 to about 20 wt. %, about 1 to about 15 wt. %, about 1 to about 10 wt. %, about 1 to about 7 wt. %, about 1 to about 5 wt. %; from about 5 to about 30 wt. %, about 5 to about 25 wt. %, about 5 to about 20 wt. %, about 5 to about 15 wt. %, about 5 to about 10 wt. %; from about 10 to about 30 wt. %, about 10 to about 25 wt. %, about 10 to about 20 wt. %, about 10 to about 15 wt. %; from about 15 to about 30 wt. %, about 15 to about 25 wt. %, about 15 to about 20 wt. %; from about 20 to about 30 wt. %, about 20 to about 25 wt. %; from about 25 to about 30 wt. %, including ranges and subranges thereof, based on the total weight of the personal care composition. In some instances, the total amount of film-former(s) may be about 1 to about 25 wt. %, about 1 to about 20 wt. %, about 1 to about 15 wt. %, about 1 to about 10 wt. %, about 5 to about 30 wt. %, about 5 to about 25 wt. %, about 5 to about 20 wt. %, about 5 to about 5 wt. %, about 5 to about 10 wt. %, based on the total weight of the personal care composition.

The personal care compositions may include any of the following additional ingredients in an amount of from about 0.01 to about 15 wt. %, based on the total weight of the personal care composition. In some instances, the amount of additional ingredients present in the personal care composition is from about 0.01 to about 12.5 wt. %, about 0.01 to about 10 wt. %, about 0.01 to about 8 wt. %, about 0.01 to about 6 wt. %, about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 12.5 wt. %, about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 12.5 wt. %, about 0.5 to about 10 wt. %, about 0.1 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; about 0.75 to about 12.5 wt. %, about 0.75 to about 10 wt. %, about 0.75 to about 8 wt. %, about 0.75 to about 6 wt. %, about 0.75 to about 5 wt. %, about 0.75 to about 4 wt. %, about 0.75 to about 3 wt. %, about 0.75 to about 2 wt. %, about 0.75 to about 1 wt. %; about 1 to about 12.5 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 12.5 wt. %, about 3 to about 10 wt. %, about 3 to about 8 wt. %, about 3 to about 6 wt. %, about 3 to about 5 wt. %, or about 3 to about 4 wt. %, including any range or subrange therebetween, based on the total weight of the personal care composition.

The personal care composition may comprise additional ingredients including, e.g., nonionic surfactants, amphoteric surfactants, cationic surfactants, thickening agents, preservatives, emulsifiers, colorants, pigments, oils, cosmetically acceptable active agents, natural extracts, pH adjusters, or the like.

The personal care composition may comprise one or more other cosmetically acceptable active agents, such as anti-acne agents, shine control agents, anti-microbial agents, anti-inflammatory agents, anti-mycotic agents, anti-parasite agents, external analgesics, sunscreens, photoprotectors, antioxidants, keratolytic agents, surfactants, moisturizers, nutrients, vitamins, energy enhancers, anti-perspiration agents, astringents, deodorants, firming agents, anti-callous agents, and agents for skin conditioning. The cosmetically acceptable active agent may be selected for instance from, benzoyl peroxide, D-panthenol carotenoids, ceramides, polyunsaturated fatty acids, essential fatty acids, enzymes such as laccase, enzyme inhibitors, minerals, hormones such as estrogens, steroids such as hydrocortisone, 2-dimethylaminoethanol, copper salts such as copper chloride, peptides like argireline, synake and those containing copper, coenzyme Q10, amino acids such as proline, vitamins, lactobionic acid, acetyl-coenzyme A, niacin, riboflavin, thiamin, ribose, electron transporters such as NADH and FADH2, natural extracts such as from aloe vera, feverfew, oatmeal, dill, blackberry, princess tree, Picia anomala, and chicory, resorcinols such as 4-hexyl resorcinol, curcuminoids, sugar amines such as N-acetyl glucosamines, and derivatives and mixtures thereof.

Examples of vitamins that may be incorporated into the personal care composition include, but are not limited to, vitamin A, vitamin B′s such as vitamin B3, vitamin B5, and vitamin B12, vitamin C, vitamin K, and different forms of vitamin E like alpha, beta, gamma or delta tocopherols or their mixtures, and derivatives thereof.

The personal care composition may include one or more stilbenoids. Examples of stilbenoids include piceid, resveratrol, piceatannol, pterostilbene, or a combination of two or more thereof.

Natural extracts containing antioxidants that may be suitable for use in the personal care compositions, include, but not limited to, extracts containing flavonoids and isoflavonoids and their derivatives (e.g., genistein and diadzein), extracts containing resveratrol and the like. Examples of such natural extracts include grape seed, green tea, pine bark, and propolis.

In certain embodiments, the sunscreen composition may include one or more compounds suitable for enhancing the photostability of the UV filters of other ingredients in the personal care composition. Photostabilizers include, for example, diesters or polyesters of a naphthalene dicarboxylic acid.

The personal care composition may further comprise one or more colorants. The colorants may be a pigment, a dye, or mixtures thereof. Non-limiting examples of pigments include titanium dioxide, zinc oxide, kaolin, mica etc. Non-limiting examples of dyes include food dyes suitable for food, drug and cosmetic applications, and mixtures thereof. Some color agents (colorants) are known as FD&C dyes. In some embodiments, the colorants may be present in an amount ranging from about 0.0001% wt. % to about 0.4% wt. %, including all percentages and subranges therebetween, based on the total weight of the personal care composition. In further embodiments, the colorants may be present in an amount ranging from about 0.0001% wt. % to about 4% wt. %, including all percentages and subranges therebetween, based on the total weight of the personal care composition.

The personal care compositions may include additional and/or optional thickeners other than polyvinyl pyrrolidone. Illustrative additional or optional thickeners other than polyvinyl pyrrolidone may be or include, but are not limited to, carbomers (e.g., carboxyvinyl polymers), carrageenans (e.g., Irish moss, carrageenan, iota-carrageenan, etc.), high molecular weight polyethylene glycols (e.g., CARBOWAX.RTM., which is commercially available from The Dow Chemical Company of Midland, Mich.), cellulosic polymers, hydroxyethylcellulose, carboxymethylcellulose, and salts thereof (e.g., CMC sodium), natural gums (e.g., karaya, xanthan, gum arabic, and tragacanth), colloidal magnesium aluminum silicate, and the like, and mixtures or combinations of two or more thereof. In one embodiment, the personal care composition includes a thickening system comprising a polymer selected from polyvinyl pyrrolidone, a polyacrylate, a polymethacrylate, a polyitaconate, an acrylamide, 2-acrylamido-2-methylpropane sulfonic acid (AMPS); and a combination of two or more thereof.

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, and the like. The personal care composition may include one or more bases, such as sodium hydroxide, potassium hydroxide and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final personal care composition and/or product. For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the personal care composition. The personal care compositions may have a pH from 3 to about 11.

In at least one aspect, provided is a method for protecting a skin surface from ultraviolet light; treating, inhibiting or preventing sunburn; and/or ameliorating a symptom associated with excessive sun exposure; comprising: administering an personal care composition disclosed herein to a skin surface of a subject in need thereof.

In at least one other aspect, provides is a use of a composition comprising an amino acid complex for protecting a skin surface from ultraviolet light; treating, inhibiting or preventing sunburn; and/or ameliorating a symptom associated with excessive sun exposure. The symptom associated with excessive sun exposure is selected from: inflammation; itch; hypersensitivity; peeling; rash; chills; burning; pain; blisters; sores; and flaking.

According to a further aspect of the invention, provided is a method for improving health and/or appearance of keratin (e.g., skin), the method comprising applying a first personal care composition being a sunscreen composition and applying a second personal care composition containing an amino acid complex, optionally one or more antioxidants, optionally a penetration enhancer, and optionally a cosmetically acceptable carrier. Although the sunscreen composition may be applied before the second personal care composition, in some instances the second personal care composition is applied before or simultaneously with the sunscreen composition. For example, the second personal care composition may be applied 5 minutes or more, 30 minutes or more, 1 hour or more, 2 hours or more, etc. after application of the sunscreen composition. Without being limited to any particular theory, it is believed that a synergistic improvement of skin health is achieved when a personal care composition having an amino acid complex, optionally one or more antioxidants, and optionally a penetration enhancer is applied and a sunscreen composition is applied to the same portion of skin. As noted above, the sunscreen composition may be applied first, simultaneously, or after the application of the personal care composition containing the amino acid complex.

According to some embodiments, the personal care composition containing the amino acid complex may be applied before or after the sunscreen compositions within about 1 second to about 12 hours, about 1 second to about 10 hours, about 1 second to about 8 hours, about 1 second to about 7 hours, about 1 second to about 6 hours, about 1 second to about 5 hours, about 1 second to about 4 hours, about 1 second to about 3 hours, about 1 second to about 2 hours, about 1 second to about 1 hour, about 1 second to about 45 minutes, about 1 second to about 30 minutes; from about 1 minute to about 12 hours, about 1 minute to about 10 hours, about 1 minute to about 8 hours, about 1 minute to about 7 hours, about 1 minute to about 6 hours, about 1 minute to about 5 hours, about 1 minute to about 4 hours, about 1 minute to about 3 hours, about 1 minute to about 2 hours, about 1 minute to about 1 hour, about 1 minute to about 45 minutes, about 1 minute to about 30 minutes; from about 15 minutes to about 12 hours, about 15 minutes to about 10 hours, about 15 minutes to about 8 hours, about 15 minutes to about 7 hours, about 15 minutes to about 6 hours, about 15 minutes to about 5 hours, about 15 minutes to about 4 hours, about 15 minutes to about 3 hours, about 15 minutes to about 2 hours, about 15 minutes to about 1 hour, about 15 minutes to about 45 minutes; from about 30 minutes to about 12 hours, about 30 minutes to about 10 hours, about 30 minutes to about 8 hours, about 30 minutes to about 7 hours, about 30 minutes to about 6 hours, about 30 minutes to about 5 hours, about 30 minutes to about 4 hours, about 30 minutes to about 3 hours, about 30 minutes to about 2 hours, about 30 minutes to about 1 hour; from about 45 minutes to about 12 hours, about 45 minutes to about 10 hours, about 45 minutes to about 8 hours, about 45 minutes to about 7 hours, about 45 minutes to about 6 hours, about 45 minutes to about 5 hours, about 45 minutes to about 4 hours, about 45 minutes to about 3 hours, about 45 minutes to about 2 hours; from about 1 hour to about 12 hours, about 1 hour to about 10 hours, about 1 hour to about 8 hours, about 1 hour to about 7 hours, about 1 hour to about 6 hours, about 1 hour to about 5 hours, about 1 hour to about 4 hours, about 1 hour to about 3 hours, about 1 hour to about 2 hours; from about 2 hours to about 12 hours, about 2 hours to about 10 hours, about 2 hours to about 8 hours, about 2 hours to about 7 hours, about 2 hours to about 6 hours, about 2 hours to about 5 hours, about 2 hours to about 4 hours; from about 4 hours to about 12 hours, about 4 hours to about 10 hours, about 4 hours to about 8 hours, about 4 hours to about 7 hours, or about 4 hours to about 6 hours, the foregoing ranges being inclusive of the endpoints.

### EXAMPLES

### Example

A non-limiting, example composition (Ex. A) was prepared in accordance with aspects of the invention. A comparative composition (Comp. Ex. 1) was also prepared having a similar formulation to Ex. A, except that Comp. Ex. I did not include the amino acid mixture, tocopheryl acetate, vitamin E, or sodium ascorbyl phosphate. The formulations for Ex. A and Comp. Ex. 1 are shown in Table 1.

TABLE 1

| | Comp. | |
|---|---|---|
| | Ex. 1 | Ex. A |
| Ingredient | Wt. % | |
| WATER | Q.S. | Q.S. |
| PROPYLENE GLYCOL | 5-10 | 5-10 |
| ISOPROPYL PALMITATE | 5-10 | 5-10 |
| PETROLATUM JELLY WHITE | 1-10 | 1-10 |
| GLYCERYL MONOSTEARATE | 1-8 | 1-8 |
| PEG-100 STEARATE | 1-5 | 1-5 |
| CETYL ALCOHOL | 1-5 | 1-5 |
| POLYDIMETHYLSILOXANE (350 CTS) | 0.1-3 | 0.1-3 |
| SODIUM BENZOATE | 0.1-1 | 0.1-1 |
| LACTIC ACID | 0.1-1 | 0.1-1 |
| CAPRYLYL GLYCOL | 0.1-1 | 0.1-1 |
| GLYCERIN | 0.1-1 | 0.1-1 |
| TAURINE | — | 0.65 |
| L-ARGININE | — | 0.34 |
| GLYCINE | — | 0.1 |
| TOCOPHERYL ACETATE, VITAMIN E, and | — | 0.1-1 |
| SODIUM ASCORBYL PHOSPHATE | | |
| PENTAERYTHRITYL TETRA-DI-T-BUTYL | — | 0.01-0.5 |
| HYDROXYHYDROCINNAMATE | | |

### Example 2

A UV irradiation study was conducted to assess the level of UV protection provided by Ex. A in comparison to Comp. Ex. 1 (which did not contain the combination of ingredients according to aspects of the invention). Specifically, fibroblasts cells were grown in Dulbecco's modified Eagle's medium and supplemented with 10% of fetal calf serum, 40 mg/l of gentamicin and 2 mg/l of fungizone (DMEMc), in an incubator at a temperature of 37° C. with 5 vol. % of CO₂ and 95 vol. % of air. A composition was prepared containing a mixture of taurine, L-arginine, and glycine.

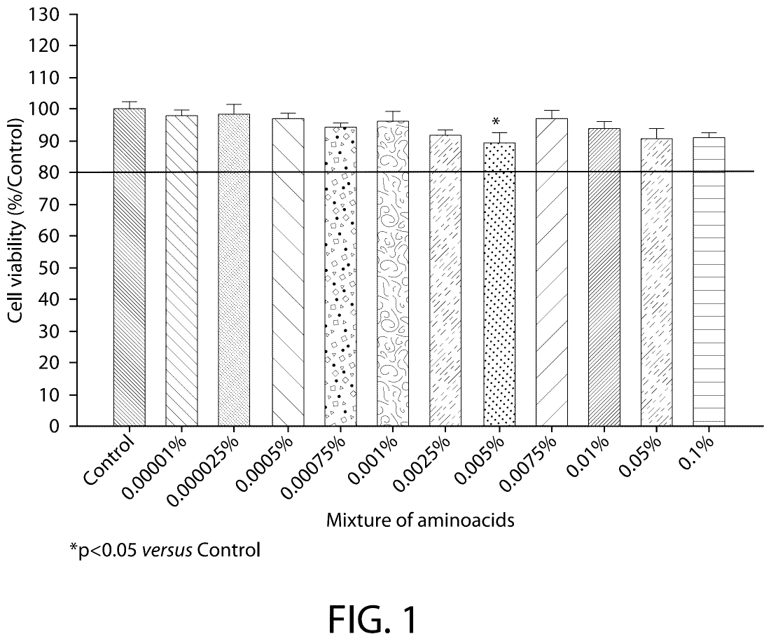

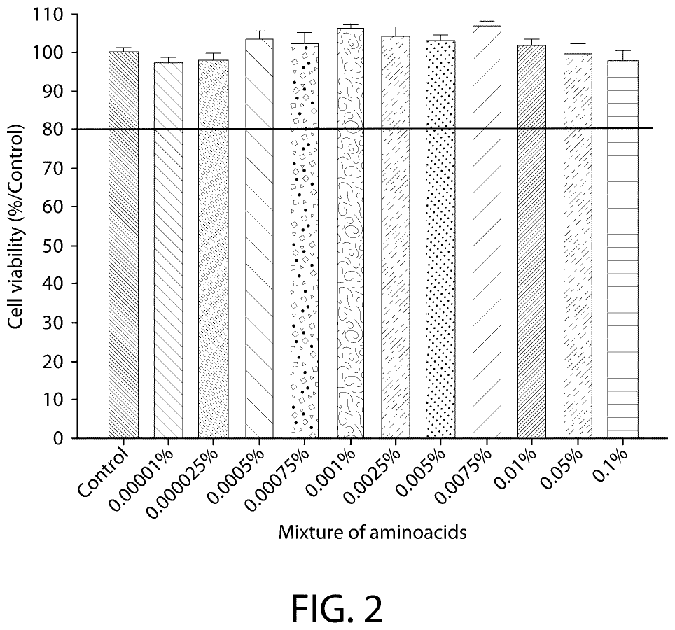

The fibroblast cells were separated into groups with one group receiving 0.1 wt. % of the amino acid mixture, one group receiving the 0.1 wt. % of the amino acid mixture and UVA irradiation at 3 J/cm², one group receiving UVA irradiation at 3 J/cm², and a negative control group, which did not receive the composition containing the amino acid mixture or the UVA irradiation. Cytotoxicity quantification was obtained from the fibroblast cells by MTT assay. Various concentrations of the amino acid mixture were evaluated after 24 hours (h) and 48 h (see FIGS. 1 and 2, respectively). A GlasBoxPlus device was used to measure collagen I synthesis through contractile forces of fibroblasts.

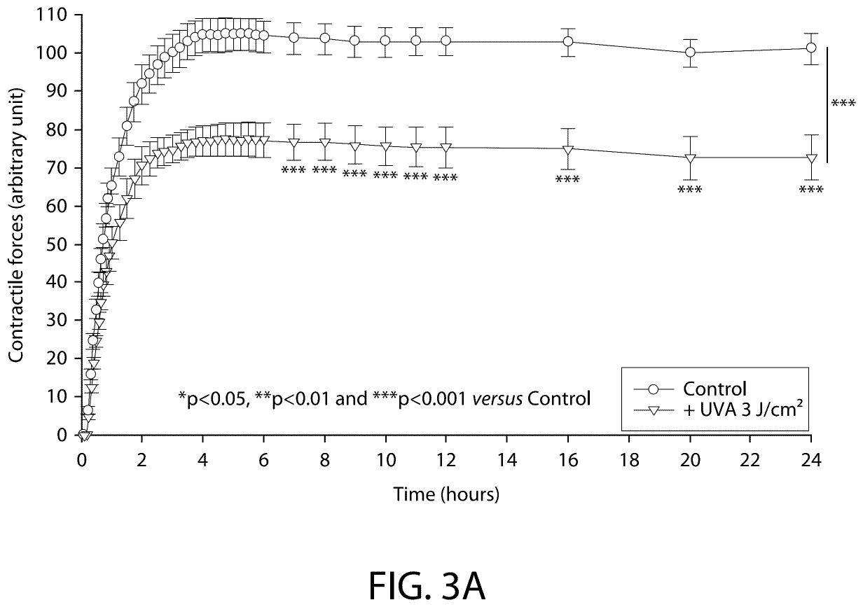

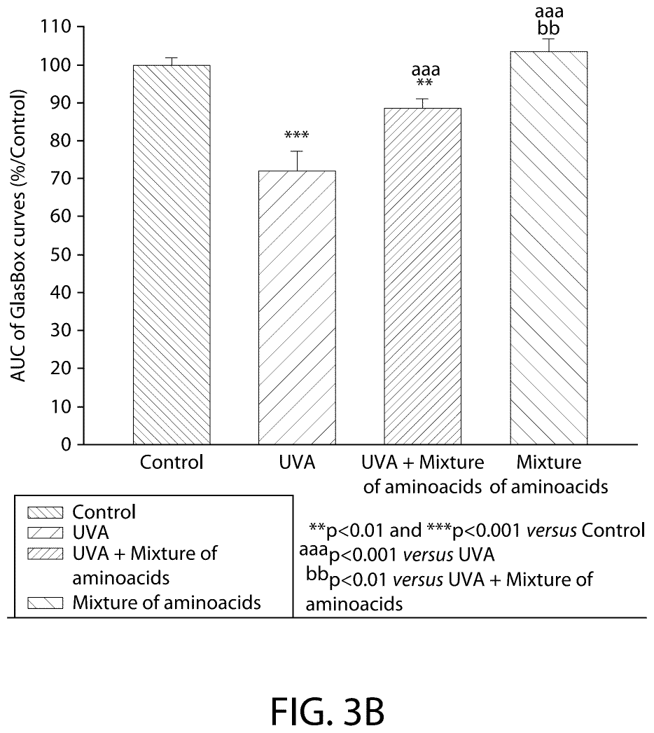

The fibroblast cytotoxicity results after 24 and 48 hours (see FIGS. 1 and 2) reveal that the amino acid mixture did not negatively affect cell viability at any of the tested concentrations. As displayed in FIGS. 3A and 3B, UV irradiation reduced the contractile force of fibroblast cells. The results of FIGS. 3A and 3B demonstrate that the mixture of amino acids provides significant recovery of the contractile force of fibroblast cells, close to the initial contractile force before UV treatment of the fibroblast cells.

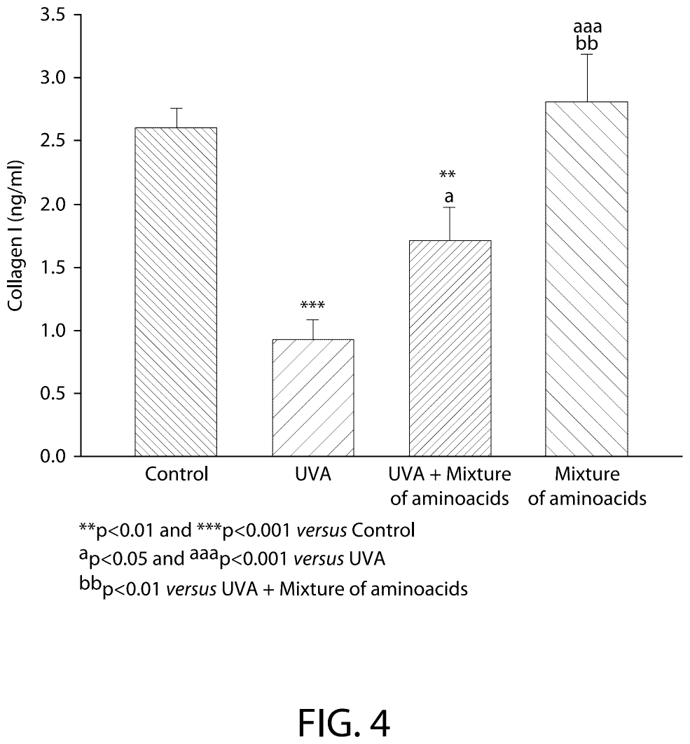

Collagen I synthesis was measured by enzyme-linked immunosorbent assay (ELISA) and the results showed a very similar trend (see FIG. 4). In particular, while the UV irradiation significantly reduced the amount of collagen, the mixture of amino acids brought the amount of collagen back to initial amounts.

### Example 3

A UV study was conducted to assess the effect of Example Composition A on a skin explant. In particular, Ex. A, Comp. Ex. 1, and a benchmark hydrocortisone cream containing 0.127% of hydrocortisone (Comp. Ex. 2), were applied to respective ex vivo skin explants. The ex vino skin explants were obtained from the abdomen of a human volunteer during a surgery procedure and placed immediately in phosphate buffer at a temperature of 37° C. The ex vivo skin explants were evaluated pursuant the following groups:
Application of Ex. A without UVA irradiation;
Application of Ex. A with UVA irradiation;
Comp. Ex. 1 without UVA irradiation;
Comp. Ex. 1 with UVA irradiation;
Comp. Ex. 2 without UVA irradiation; and
Comp. Ex. 2 with UVA irradiation.

The microdialysis system consisted of a CMA/100 syringe pump and CMA/140 microfraction collector, which collected samples. Six probes were inserted into dermis of each segment of ex vivo skin explant and perfused with Ringer solution at 3 ul/min. After one hour of stabilization, the microdialysis was started for 1 hour (T0). UVA irradiation was started using an UVA Bridge and performed during 4 hours corresponding to 17 J/cm².

Immediately after the irradiation, 2 mg/cm² of Ex. A, Comp. Ex. 1, and Comp. Ex. 2 were applied on the surface of respective segment of ex vivo skin explant. Microdialysis samples were collected every hour for 24 h. Microdialysis samples pooled at 1 h, 2 h, 4 h, 6 h, 12 h and 24 h and frozen at a temperature of-80° C. until analysis. Three biomarkers—namely, TNFα, histamine, and MMP-1—were evaluated. Histamine was quantified by the enzyme immunoassay, while TNFα and MMP-1 were determined using an ELISA kit.

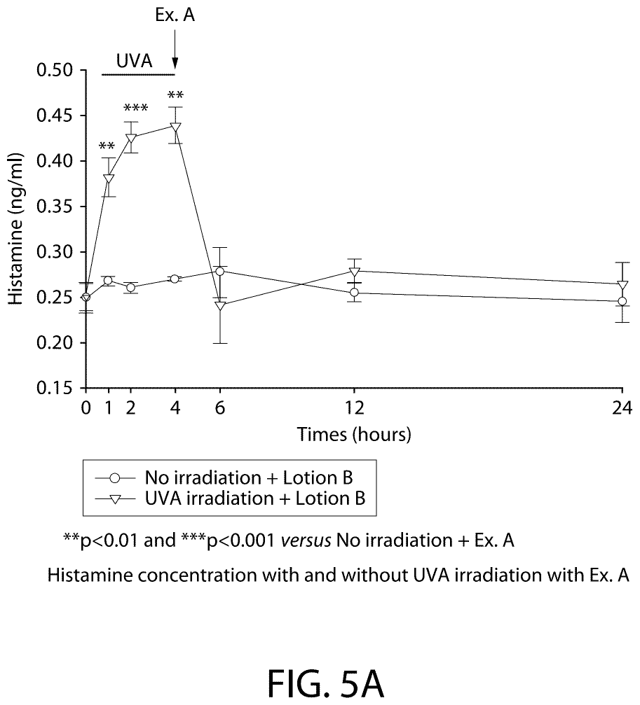

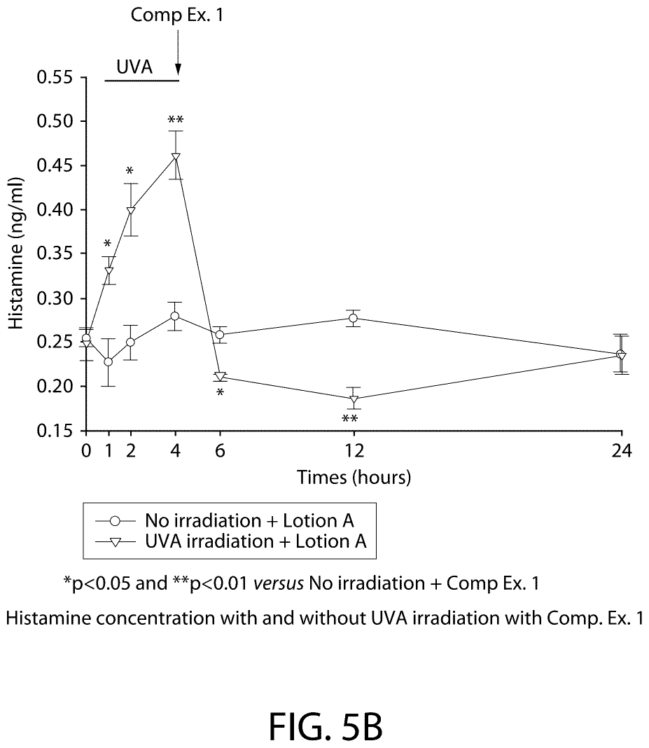

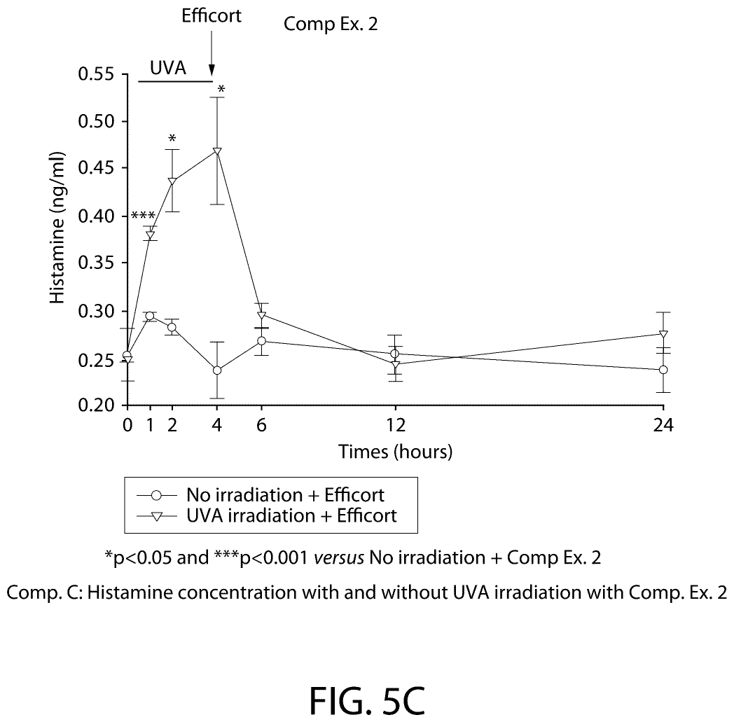

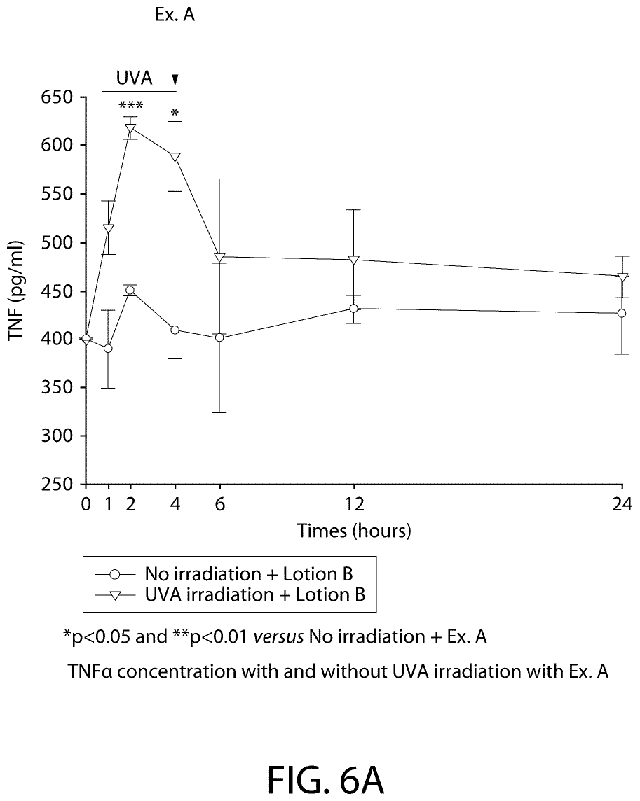

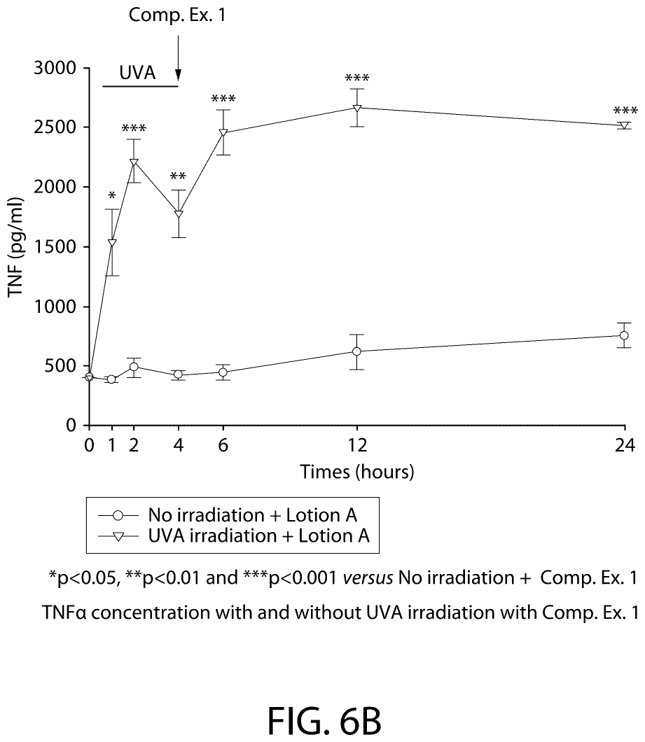

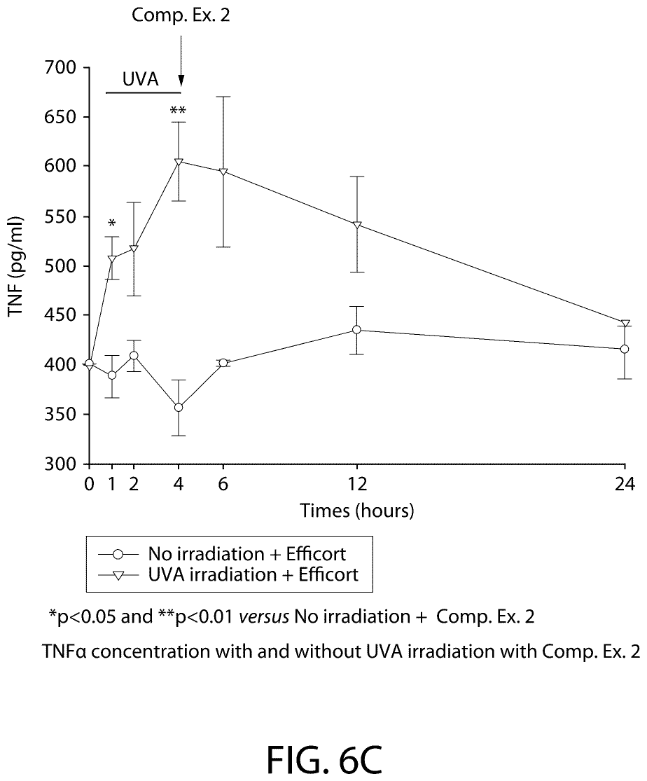

All three compositions provided a reduction of histamine and showed an allergen biomarker after UV treatment, as shown in FIGS. 5A-5C. The ex vivo skin plant receiving Ex. A exhibited a significant reduced amount of TNFα at the 6-hour time point, even faster than Comp. Ex. 2, which contained hydrocortisone (see FIG. 6A and FIG. 6C). The placebo lotion (Comp. Ex. 1) showed no reduction in TNFα for the whole time period (see FIG. 6B).

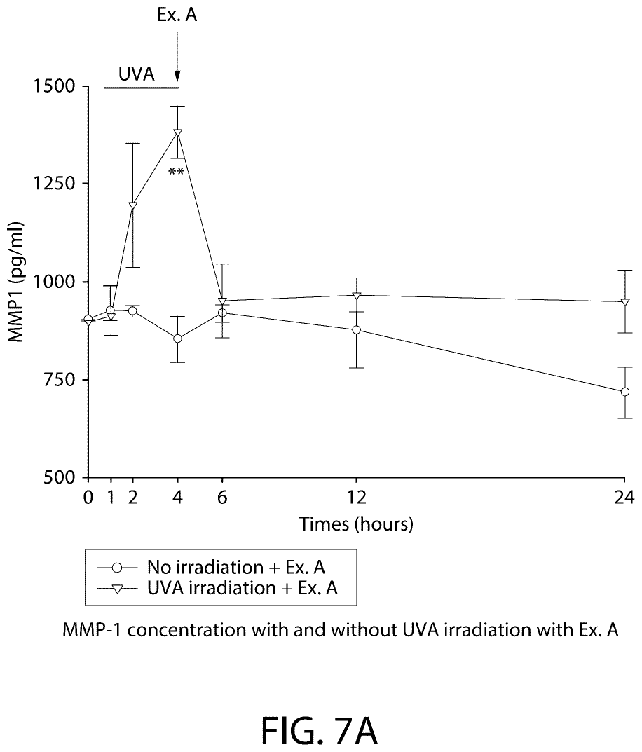

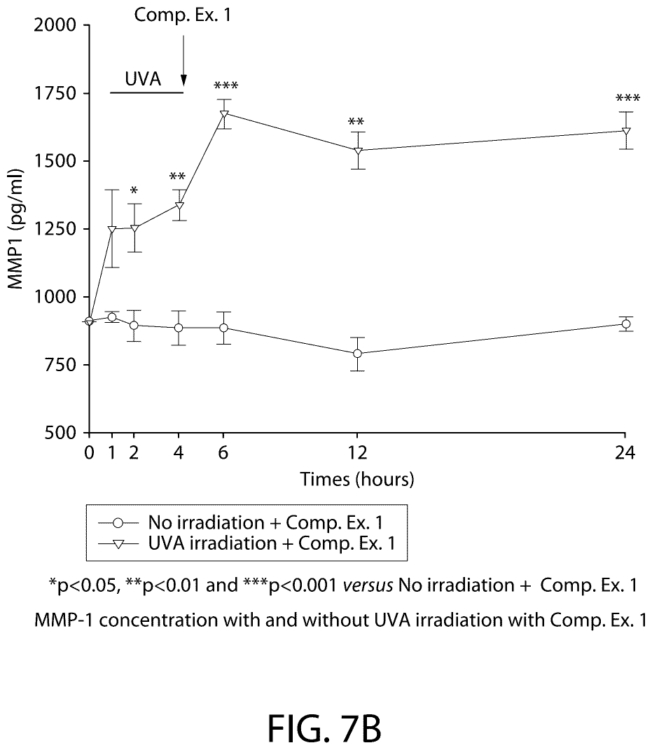

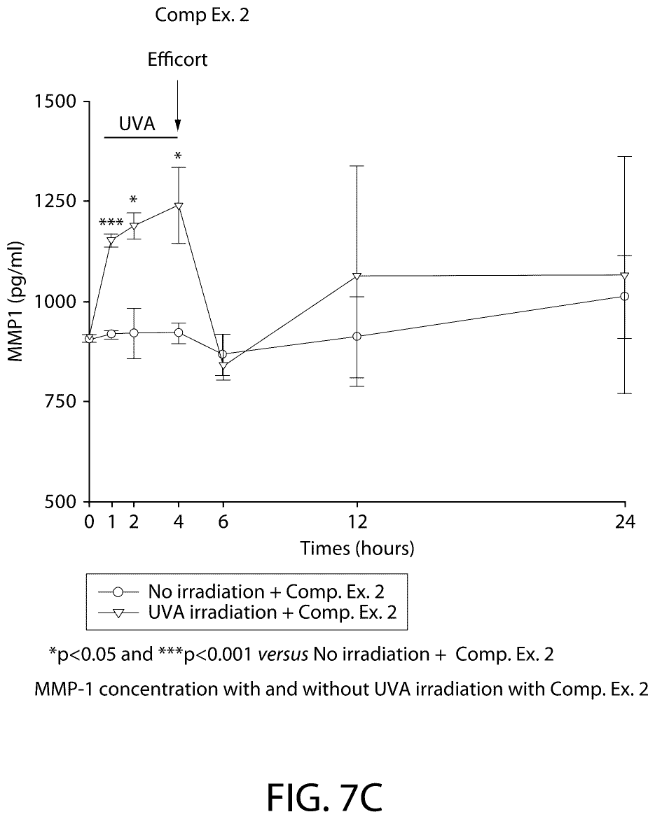

Additionally, both Ex. A and Comp. Ex. 2 significantly reduced the level of MMP-1, while Comp. Ex. I did not reduce the level of MMP-1. MMP-1 breaks down collagen in the skin. Graphs of the level of MMP-1 over time for the ex vivo explants are shown in FIGS. 7A-7C.

Based on the foregoing Examples, the application of UVA reduces fibroblasts cell contractile force and collagen synthesis. The application of compositions containing the amino acid mixture according to aspects of the invention reduced the negative effects of UVA on cell contractile force and collagen synthesis. Additionally, Ex. A, which contained the amino acid mixture in conjunction with vitamins E and C, surprisingly provided effects similar to a hydrocortisone cream, Comp. Ex. 2, for allergen, irritation, and anti-aging biomarkers.

### Example 4

A non-liming exemplary serum containing an amino acid mixture comprising taurine, L-arginine, and glycine (Ex. B) was applied to artificial skin to assess the skin barrier formed by the serum. The segments of artificial skin were artificial human epidermis obtained from MATTEK under the product name, EPIDERM. The segments of artificial skin were equilibrated overnight at a temperature of 37° C. before the treatment of UVA and UVB irradiation. A solar simulator—specifically a LS-1000 solar simulator commercially available from SOLAR LIGHT—was used to apply specific doses of UVA and UVB irradiation.

To evaluate Ex. B, segments of artificial skin received an application of Ex. B after the UV dosing for 24 hours. When sunscreen was applied to the segments of artificial skin, a broad-spectrum SPF 46 sunscreen (Comp. Ex. 3) was applied one hour prior to the UV dosing. A positive control was produced by exposing segments of the artificial skin to the UV dosing without applying Ex. B or Comp. Ex. 3. A negative control was obtained by assessing segments of artificial skin that did not receive UV dosing, Ex. B, or Comp. Ex. 3. Table 2, below, provides a summary of the evaluated segments of artificial skin.

TABLE 2

| | Comp. | Solar | |
|---|---|---|---|
| | Ex. 3 | (UVA + UVB) | Ex. B |
| Untreated | | | |
| Solar | | X | |
| Solar + SR Serum | | X | X |
| UV Clear + Solar + SR Serum | X | X | X |

For immunostainings, a 10 J/cm² dose was used for the study. For gene expression analysis and protein analysis, 20 J/cm² dose was used. After 24 hours of treatment, media was collected for IL-1a ELISA and tissues were harvested for gene expression or protein expression analysis.

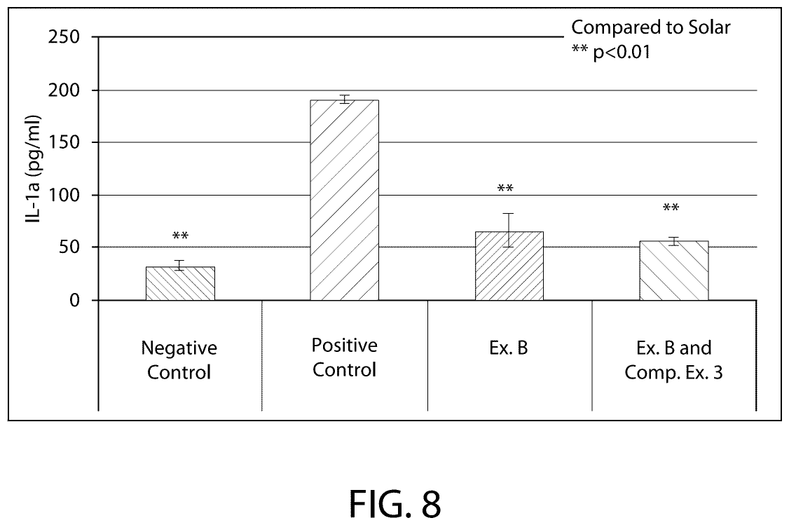

As seen in FIG. 8, after one dose of 20 J/cm² of UVA and UVB, skin irritation biomarker, IL-1a, was induced significantly. However, the segments of artificial skin receiving Ex. A resulted in the reduction of skin irritation, which was statistically significant (p value<0.01). Pre-treatment with Comp. Ex. 3 significantly reduced skin irritation as well.

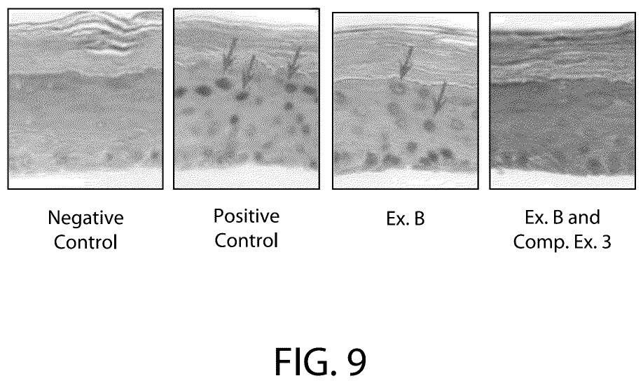

In order to assess UVB induced changes, harvested tissues were fixed and processed for paraffin sections. Thymine dimer is a hallmark of UVB irradiation, indicating two adjacent thymines forming a covalent bond. Thymine dimers need to be repaired, which might result in the development of skin cancer. The negative control, which did not receive UVA or UVB irradiation did not show any thymine dimer presence as well as in the sunscreen pretreated samples (see FIG. 9).

The positive control, which received UVA and UVB at a dose of 10 J/cm², however, exhibited increased thymine dimer formation (red arrows) in the nucleus. The artificial skin that received Ex. B resulted in lighter, more hollow thymine dimer staining, which indicates recovery from thymine dimer formations.

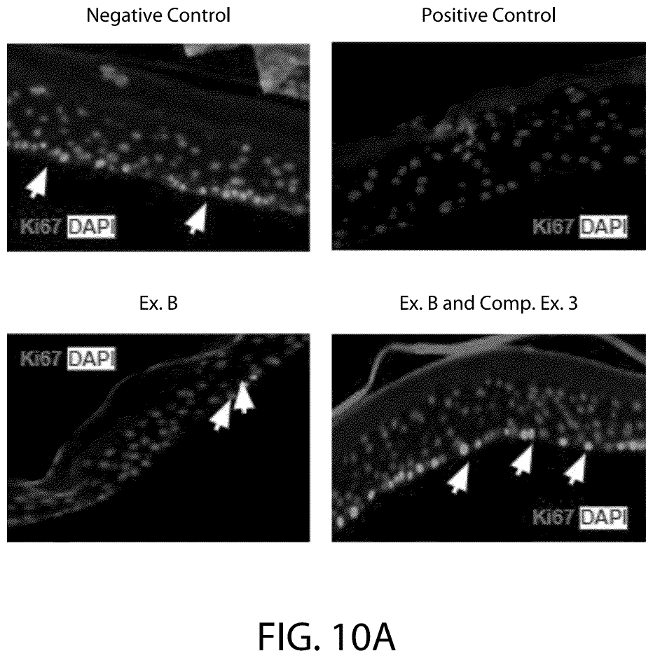

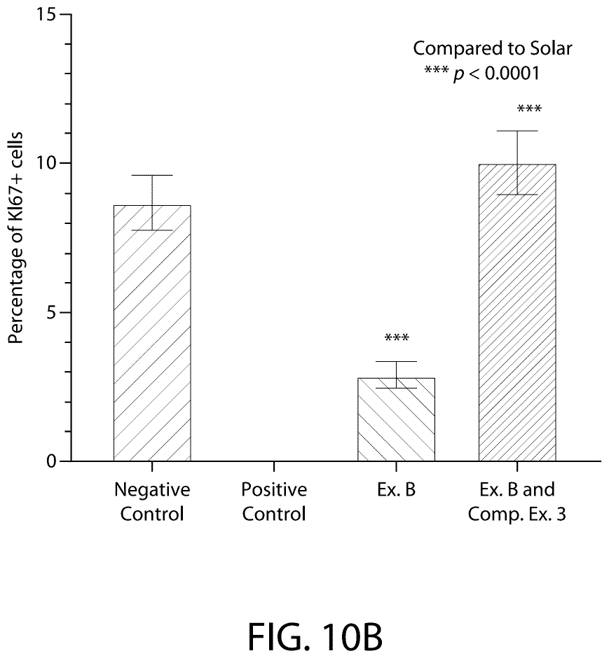

The same sets of tissues were subsequently stained for Ki67, a keratinocyte proliferation biomarker. The immunofluorescence results are shown in FIG. 10A and the quantitation was graphed in FIG. 10B. UVA and UVB irradiation for the positive control resulted in complete reduction of Ki67 positive cells, while treatments with Ex. B after UV irradiation resulted in some positive nucleuses indicating the recovery process. Prior treatment with Comp. Ex. 3 prevented the loss of Ki67 positive cells.

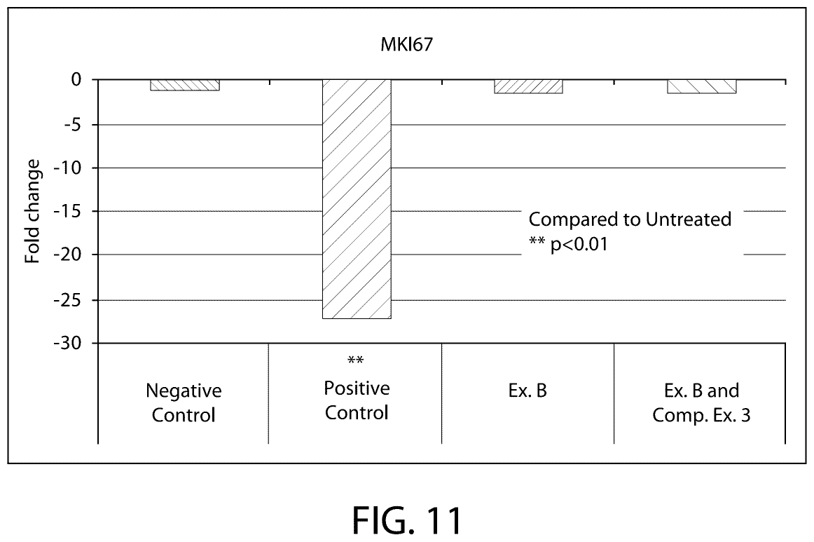

Regarding the gene expression analysis, UV irradiation resulted in over 25-fold inhibition of MKI67 (Marker for Ki67), as seen for the positive control in FIG. 11. Ex. B resulted in recovery to the level of untreated artificial skin. Moreover, the results of Ex. B were surprisingly similar to pre-treatment with Comp. Ex. 3.

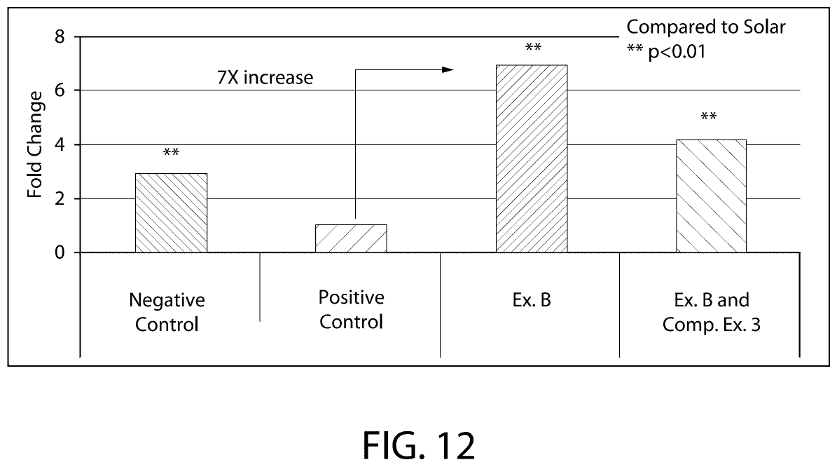

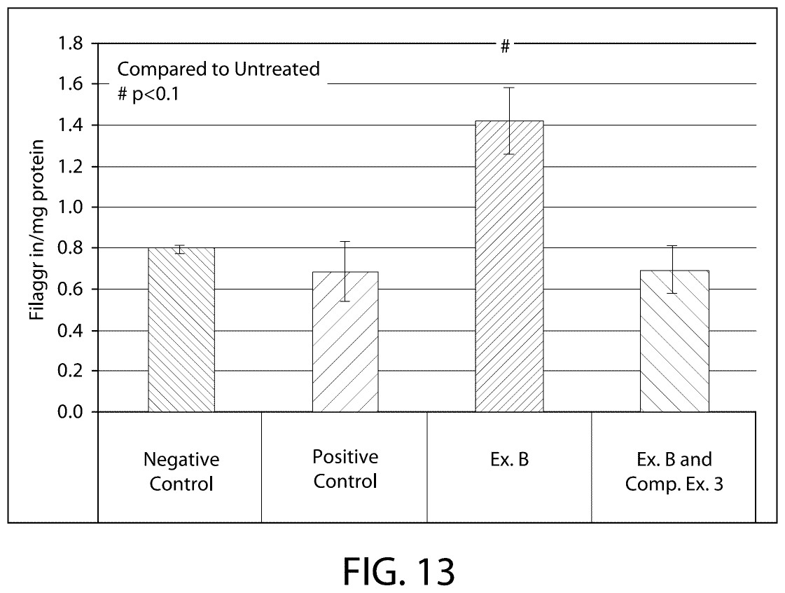

Filaggrin expression, a skin barrier biomarker, was assessed by QPCR (see FIG. 12) and ELISA (see FIG. 13). While UV irradiation suppressed filaggrin gene expression by 3-fold in the positive control, application of Ex. B after UV irradiation resulted in increased filaggrin expression by 7-fold, which was statistically significant. Protein expression by ELISA did not show 7-fold increase, however the application of Ex. B after UV irradiation resulted in higher filaggrin protein expression.

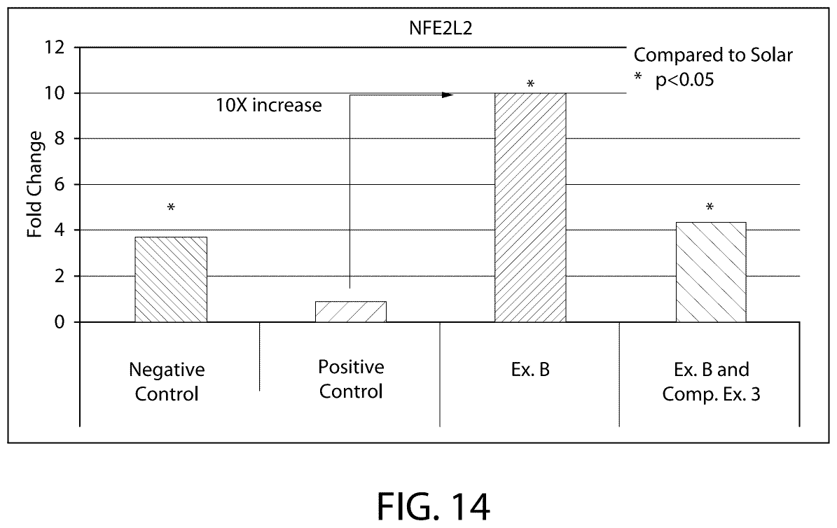

The antioxidant regulator gene, NFE2L2, was also downregulated by UV irradiation by almost 4-fold, as seen for the positive control in FIG. 14. However, the application of Ex. B after UV irradiation resulted in a 10-fold increase in NFE2L2 expression, which suggests oxidative stress from UV irradiation was being restored and rebuilt.

In view of the foregoing results, it was surprising that Ex. B provided significant benefits in reducing the impact of UV induced changes in skin. UV irradiation increased skin irritation, increased thymine dimer formation, reduced cell proliferation, reduced skin barrier and antioxidant regulator expression. All these biomarkers were restored or partially restored after application/treatments with Ex. B, which demonstrate the recovery/repair potential of certain embodiments of the invention for UV induced damages in the skin.

### Example 5

A non-limiting, example personal care composition was prepared in accordance with aspects of the invention and evaluated to assess the effects of such exemplary personal care composition in comparison to a conventional sunscreen composition.

Several samples of artificial skin were prepared to assess the exemplary personal care compositions. A control (Comp. Ex. 3) was prepared from a sample of artificial skin that was not exposed to UV radiation and did not receive either the sunscreen composition or the exemplary compositions. A second control (Comp. Ex. 4) was prepared from a sample of the artificial skin by exposing the sample to UVA and UVB radiation at 10 J/cm² for 10 minutes. A comparative sample of artificial skin (Comp. Ex. 5) was prepared by applying a conventional broad-spectrum SPF 46 sunscreen composition to the sample of artificial skin 1 hour before the sample was exposed to UVA and UVB radiation at 10 J/cm² for 10 minutes.

The list of active and inactive ingredients for the broad-spectrum SPF 46 sunscreen composition used to prepare Comp. Ex. 3 is provided below.
Active Ingredients: 9.0% Zinc oxide, and 7.5% Octinoxate.
Inactive Ingredients: Purified Water, Cyclomethicone, Niacinamide, Octyldodecyl Neopentanoate, Hydroxyethyl Acrylate/Sodium Acryloyldimethyl Taurate Copolymer, Polyisobutene, PEG-7 Trimethylolpropane Coconut Ether, Sodium Hyaluronate, Tocopheryl Acetate, Lactic Acid, Oleth-3 Phosphate, Phenoxyethanol, Butylene Glycol, Iodopropynyl Butylcarbamate, and Triethoxycaprylylsilane.

An exemplary sample of artificial skin (Ex. C) was prepared by applying an exemplary personal care composition comprising an amino acid complex of taurine, L-arginine, and glycine according to aspects of the invention after the sample of artificial skin was exposed to UVA and UVB radiation at 10 J/cm² for 10 minutes. Another exemplary sample of artificial skin (Ex. D) was prepared by applying the same conventional broad-spectrum SPF 46 sunscreen composition as Comp. Ex. 5 to a sample of artificial skin 1 hour before being exposed to UVA and UVB radiation at 10 J/cm² for 10 minutes and then applying the same personal care composition as Ex. C to the sample of artificial skin. The list of ingredients for the exemplary personal care compositions used to prepare Ex. C is provided below:
Water, Propanediol, Isododecane, Glycerin, Dimethicone, Polymethylsilsesquioxane, Polysilicone-11, Panthenol, Coco-Caprylate/Caprate, Taurine, Sodium Acrylate/Sodium, Acryloyldimethyl Taurate Copolymer, Arginine, Butylene Glycol, Tocopherol, Hydroxyacetophenone, Isohexadecane, Centella Asiatica Leaf Extract, Ammonium Acryloyldimethyltaurate/Carboxyethyl Acrylate Crosspolymer, Caprylyl Glycol, 1,2-Hexanediol, Coco-Glucoside, Polysorbate 80, Decyl Glucoside, Hydroxyethylcellulose, Citric Acid, and Trisodium Ethylenediamine Disuccinate, Glycine, and Sodium Hyaluronate.

In vitro studies were conducted using MatTek's patented EpiDerm with an in vitro tissue system. The artificial skin was normalized with tissue culture media by overnight incubation at a temperature of 37° C. with 5% CO₂ before the application of any composition or exposure to UVA and UVB radiation. After preparing artificial skin samples as described above, each artificial skin sample was incubate for 24 hours at a temperature of 37° C. with 5% CO₂. The tissue samples were then collected, fixed with a solution containing 4 wt. % paraformaldehyde (PFA), followed by a paraffin embedding processes. PFA-fixed paraffin-embedded mouse intestinal tissue sections were de-waxed by heat and xylene treatment followed by hydration of the tissue by a gradient of ethanol ranging from 100 wt. % to 50 wt. % followed by water. The tissue slides then went through the antigen retrieval step using citric acid-based solution (pH 6) for 30 minutes, during which tissue slides were immersed in the solution and heated using a commercially available hot plate stirrer. The tissue slides were then cooled down to a temperature 60° C., and washed 1X phosphate buffered saline (PBS). The artificial samples were subsequently blocked for 1 hour 30 minutes at room temperature in PBS containing 5% goat serum and 0.1% triton x 100. Primary antibody (anti-rabbit Ki67 (SP6), abcam 16667, 1:200) incubations proceeded overnight at a temperature of 4° C. For post primary antibody treatment, the tissue slides were washed in PBS and incubated with fluorescently labelled secondary antibody (Alexa Fluor 488 Dye, ThermoFisher Scientific, A11006, 1:1000) for 1 hour 30 minutes at room temperature. Following the secondary antibody treatment, slides were washed with PBS followed by air-drying and mounting them with Prolong Gold antifade medium containing DAPI nuclear counterstain (vectashield antifade mounting media with DAPI) (vector laboratories, H1200). Images were collected by microscope, EVOS FL Auto (Life technologies).

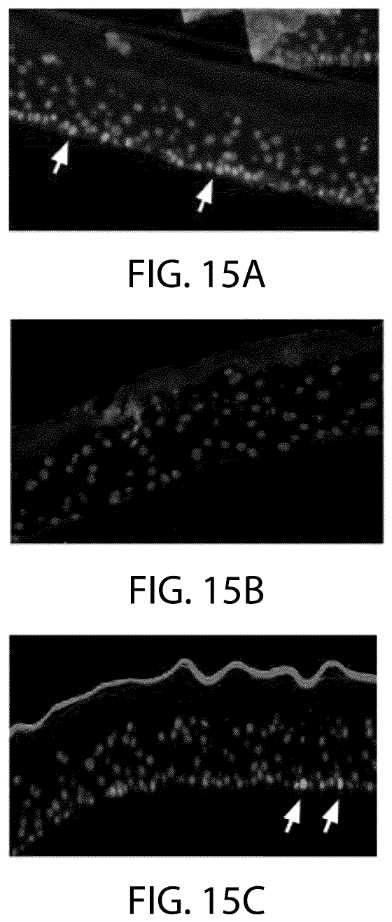

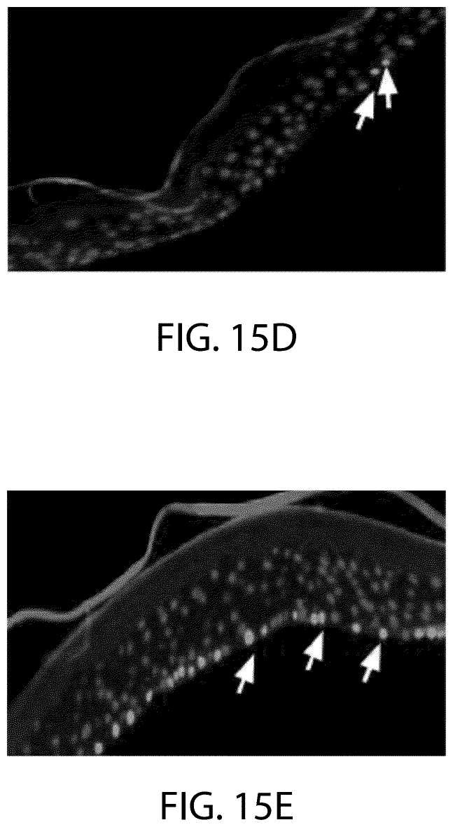

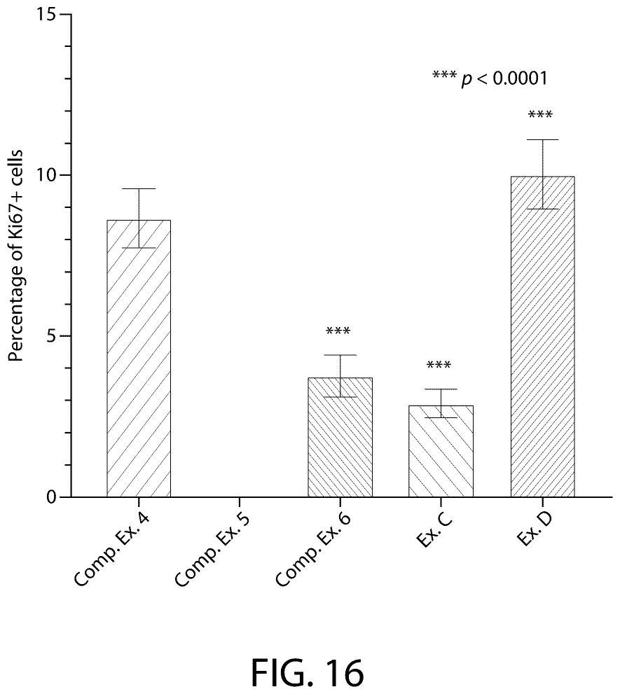

The results of the Ki67 expression are shown in Table 3 and FIGS. 15A-16.

TABLE 3

| | | | % Increase |
|---|---|---|---|
| | | | (compared to |
| | Treatment | P values | UVA + UVB) |
| A vs B | Untreated Vs UVA + UVB only | ***p < 0.0001 | 0% |
| B vs C | UVA + UVB only Vs Broad-Spectrum | *** p = 0.0004 | 375% |
| | SPF 46 sunscreen + UV | | |
| B vs D | UVA + UVB only Vs EltaMD Skin | *** p = 0.0002 | 289% |
| | Recovery Serum + UV | | |
| B vs E | UV (A + B) Vs Broad-Spectrum | ***p < 0.0001 | 1002% |
| | SPF 46 sunscreen + UV + EltaMD | | |
| | Skin Recovery Serum | | |

As seen in FIG. 16 and Table 3, the artificial skin samples receiving Ex. D exhibited significantly more Ki67 expression than the combination of Comp. Ex 6 and Ex. C, which was unexpected.

To evaluate the IL-1α, the media surrounding the in-vitro epidermal tissues were collected. All reagents and working standards were prepared as per directions on the Recombinant Human IL-1α/IL-1F1 Protein ELISA kit R&D systems. 50 μl of the assay diluent was added to each well followed by 200 μl of standard and samples in the respective wells. The plate was then incubated for 2 hours at room temperature. The wells were then washed with wash buffer and 200 μl of IL-1α conjugate was added to each well and incubated for 1 hour at room temperature. The wells were then washed with a wash buffer and 200 μl of substrate solution was added to each well, incubated for 20 minutes at room temperature while being protected from light. Following the incubation, 50 μl of stop buffer solution was added to each well and optical density of each well was determined using a microplate reader set at 450 nm.

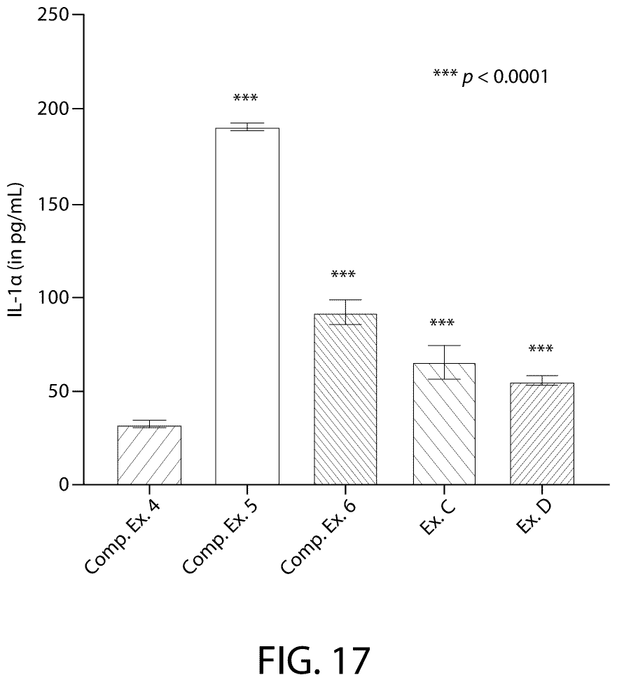

The results of the IL-1α expression are shown in Table 4 and FIG. 17.

TABLE 4

| | | | % Increase |
|---|---|---|---|
| | | | (compared |
| | | | to UV |
| | Treatment | P values | (A + B) |
| A vs B | Untreated Vs UV (A + B) | ***p < 0.0001 | 0% |
| B vs C | UV (A + B) Vs Broad- | *** p = 0.0001 | 51.7% |
| | Spectrum SPF 46 | | |
| | sunscreen + UV | | |
| B vs D | UV (A + B) Vs EltaMD Skin | *** p = 0.0002 | 65.7% |
| | Recovery Serum + UV | | |
| B vs E | UV (A + B) Vs Broad- | ***p < 0.0001 | 71% |
| | Spectrum SPF 46 sunscreen + | | |
| | UV + EltaMD Skin Recovery | | |
| | Serum | | |

The results demonstrated that simultaneous application of the conventional broad-spectrum SPF 46 sunscreen and the exemplary personal care composition (containing the amino acid complex), when applied onto UV damaged skin protected and restored the skin to its untreated/nascent form by reducing skin's irritation (decreasing IL-1α protein release) and restoring its cellular proliferation (increasing Ki67 expression). An equivalent combination of the broad-spectrum SPF 46 sunscreen and the exemplary personal care composition applied on skin damaged by UVA and UVB radiation, demonstrated a synergetic and significant improvement in the skin's renewal and in restoring the skin to its nascent/unexposed condition. The inventors were surprised by the synergistic and significant improvement provided by the combination of the sunscreen composition and the personal care composition.

What is claimed is:

1. A personal care composition comprising:
an antioxidant comprising vitamin E; and
a skin protection system, comprising:
amino acid comprising taurine, arginine, and glycine;
wherein the weight ratio of the taurine to arginine to glycine is about 65:34:1;
wherein the skin protection system protects a skin surface from ultraviolet light.

2. The personal care composition according to claim 1, wherein the amino acid further comprises an amino acid selected from: serine, lysine, and a combination thereof.

3. The personal care composition according to claim 1, wherein the antioxidant is further selected from: sulfhydryl compounds; lipoic acid; dihydrolipoic acid; resveratrol; lactoferrin;
ascorbic acid; butylated hydroxytoluene; retinoids; tocopherols; tocotrienols; ubiquinone;
vitamin C; vitamin A; a derivative thereof; and a combination of two or more thereof.

4. The personal care composition according to claim 1, wherein the antioxidant is selected from: vitamin C; vitamin A; a derivative thereof; and a combination of two or more thereof.

5. The personal care composition according to claim 1, further comprising a penetration enhancer.

6. The personal care composition according to claim 5, wherein the penetration enhancer is selected from: ethanol, dimethyl sulfoxide, dimethyl isosorbide, isopropyl myristate; propylene glycol; and a combination of two or more thereof.

7. The personal care composition of claim 1 further comprising a cosmetically acceptable active agent selected from an anti-acne agent, a shine control agent, an anti-microbial agent, an anti-inflammatory agent, an anti-mycotic agent, an anti-parasite agent, an external analgesic, a keratolytic agent, a surfactant, a moisturizer, a nutrient, a vitamin, an energy enhancer, an anti-perspiration agents, an astringent, a deodorant, a firming agent, an anti-callous agent, an agent for skin conditioning, and a combination of two or more thereof.

8. The personal care composition of claim 1 further comprising one or more pH adjusters.

9. The personal care composition of claim 8, wherein the one or more pH adjusters is present in an amount of from about 0.05 to about 20 wt. %, or 0.05 to about 15 wt. %, by total weight of the personal care composition.

10. A method for protecting a skin surface from ultraviolet light, the method comprising:
applying a personal care composition to a skin surface of a subject in need thereof, the composition comprising:
one or more antioxidants comprising vitamin E; and
a skin protection system, comprising:
amino acids comprising taurine, arginine, and glycine;
wherein the weight ratio of the taurine to arginine to glycine is about 65:34:1;
wherein the skin protection system protects a skin surface from ultraviolet light.

11. The method according to claim 10, further comprising treating, inhibiting, or preventing sunburn.

12. The method according to claim 10, further comprising ameliorating a symptom associated with excessive sun exposure.

13. The method according to claim 12, wherein the symptom associated with excessive sun exposure is selected from: inflammation; itch; hypersensitivity; peeling; rash; chills; burning;
pain; blisters; sores; and flaking.

14. The method according to claim 10, wherein the amino acid further comprises an amino acid selected from: serine, lysine, and a combination thereof.

15. The method according to claim 10, wherein the antioxidant is further selected from:
sulfhydryl compounds; lipoic acid; dihydrolipoic acid; resveratrol; lactoferrin; ascorbic acid;
butylated hydroxytoluene; retinoids; tocopherols; tocotrienols; ubiquinone; vitamin C; vitamin A; a derivative thereof; and a combination of two or more thereof.

16. The method according to claim 10, wherein the antioxidant is selected from: vitamin C;
vitamin A; a derivative thereof; and a combination of two or more thereof.

17. The method according to claim 10, wherein the personal care composition further comprises a penetration enhancer.

18. The method according to claim 17, wherein the penetration enhancer is selected from:
ethanol, dimethyl sulfoxide, dimethyl isosorbide, isopropyl myristate; propylene glycol; and a combination of two or more thereof.

19. The method according to claim 10, wherein the personal care composition further comprises one or more pH adjusters.
